(19) United States
(12) Patent Application Publication
(10) Pub. No.: US 2022/0054445 A1
(43) Pub. Date: Feb. 24, 2022

(54) THERAPEUTIC METHODS AND COMPOSITIONS FOR TREATING CANCER USING 6,8-BIS-BENZYLTHIO-OCTANOIC ACID AND AN AUTOPHAGY INHIBITOR

(71) Applicants: Rafael Pharmaceuticals, Inc., Cranbury, NJ (US); Gifu University, Gifu City, Gifu (JP)

(72) Inventors: Timothy S. Pardee, Cranbury, NJ (US); Sanjeev Luther, Cranbury, NJ (US); Paul Bingham, South Setauket, NY (US); Zuzana Zachar, South Setauket, NY (US); Shawn D. Stuart, East Moriches, NY (US); Robert G.L. Shorr, Edison, NJ (US); Tamotsu Takeuchi, Gifu City, Gifu (JP); Yusuke Kito, Gifu City, Gifu (JP); Chiemi Saigo, Gifu City, Gifu (JP)

(21) Appl. No.: 17/414,399

(22) PCT Filed: Dec. 20, 2019
(86) PCT No.: PCT/US2019/067757

§ 371 Date: Jun. 16, 2021

Related U.S. Application Data
(60) Provisional application No. 62/857,115, filed on Jun. 4, 2019.
(60) Provisional application No. 62/834,472, filed on Apr. 16, 2019.
(60) Provisional application No. 62/793,665, filed on Jan. 17, 2019.
(60) Provisional application No. 62/782,926, filed on Dec. 20, 2018.

(51) Int. Cl. A61K 31/20; A61K 31/4184; A61K 31/4706; A61P 35/00; A61P 35/02

(52) U.S. Cl. CPC A61K 31/20; A61K 31/4184; A61P 35/02; A61P 35/00; A61K 31/4706

(57) ABSTRACT

The invention provides methods and compositions for treating cancer by administering to a patient in need thereof a therapeutically effective amount of 6,8-bis-benzylthio-octanoic acid and an autophagy inhibitor.

### CROSS-REFERENCE TO RELATED APPLICATIONS

This application is the national stage application of International (PCT) Patent Application Serial No. PCT/US2019/067757, filed Dec. 20, 2019, which claims the benefit of and priority to U.S. Provisional Patent Application Ser. No. 62/782,926, filed Dec. 20, 2018; U.S. Provisional Patent Application Ser. No. 62/793,665, filed Jan. 17, 2019; U.S. Provisional Patent Application Ser. No. 62/834,472, filed Apr. 16, 2019; and U.S. Provisional Patent Application Ser. No. 62/857,115, filed Jun. 4, 2019; the contents of these applications are hereby incorporated by reference in their entirety.

### FIELD OF THE INVENTION

The invention provides methods and compositions for treating cancer by administration of 6,8-bis-benzylthio-octanoic acid and an autophagy inhibitor.

### BACKGROUND

CPI-613 (6,8-bis-benzylthio-octanoic acid) is a first-in-class investigational small-molecule (lipoate analog), which targets the altered energy metabolism that is common to many cancer cells. CPI-613 has been evaluated in multiple phase I, I/II, and II clinical studies, and has been granted orphan drug designation for the treatment of pancreatic cancer, acute myeloid leukemia (AML), peripheral T-cell lymphoma (PTCL), Burkitt lymphoma and myelodysplastic syndromes (MDS).

A need exists to improve the safety and efficacy of treating cancer with CPI-613. The present invention addresses this need and provides other related advantages.

### SUMMARY

The invention provides methods and compositions for treating cancer in a human patient in need thereof by administering to the patient a therapeutically effective amount of 6,8-bis-benzylthio-octanoic acid and an autophagy inhibitor. The cancer may be relapsed or refractory. The cancer may be a lymphoma, leukemia, carcinoma, sarcoma, melanoma, myeloma, brain or spinal cord cancer, blastoma, germ cell tumor, cancer of the pancreas, colorectal cancer, myelodysplastic syndrome, or cancer of the prostate. In certain embodiments, the cancer is a lymphoma, leukemia, carcinoma, sarcoma, melanoma, or myeloma. In certain embodiments, the cancer is relapsed or refractory Hodgkin lymphoma, including relapsed or refractory Hodgkin lymphoma in a patient who has failed brentuximab vedotin and a PD-1 inhibitor, relapsed or refractory T-cell non-Hodgkin lymphoma, relapsed or refractory Burkitt's lymphoma, or high-grade B-cell lymphoma with rearrangements of MYC and BCL2 and/or BCL6.

The foregoing aspects of the invention are described in more detail, along with additional embodiments, in the detailed description below.

### BRIEF DESCRIPTION OF THE DRAWINGS

FIG. 1 depicts induction of autophagy by 6,8-bis-benzylthio-octanoic acid in K562 and MFL2 AML cells. HBSS=Hanks's balanced salt solution.

FIG. 2 depicts the in vitro treatment of K562 and OCI-AML3 cells with a combination of chloroquine and CPI-613. All concentrations listed are μM.

FIG. 3 depicts the treatment of MFL2 syngeneic tumors in C57Bl/6 mice with a combination of CPI-613 and chloroquine.

FIG. 4 depicts the in vitro treatment of MFL2 cells with a combination of metformin and CPI-613. All concentrations listed are μM. Met=Metformin, CPI=CPI-613

FIG. 5 depicts the treatment of MFL2 syngeneic tumors in C57Bl/6 mice with a combination of metformin and CPI-613 (Met+CPI).

FIG. 6 depicts the in vitro treatment of MFL2 cells with a combination of 2-deoxyglucose and CPI-613. CPI=CPI-613 (μM), 2DG=2-deoxyglucose (mM).

FIG. 7 depicts the in vitro treatment of OCI-AML3 cells and MFL2 cells with a combination of chloroquine, 2-deoxyglucose, and CPI-613.

FIG. 8 depicts the in vitro treatment of PANC-1 cells with chloroquine, hydroxychloroquine, or CPI-613 alone or in combination.

FIG. 9 depicts the in vitro treatment of AsPC-1 cells with chloroquine, hydroxychloroquine, or CPI-613 alone or in combination.

FIG. 10 depicts the in vitro treatment of BxPC-3 cells with chloroquine, hydroxychloroquine, or CPI-613 alone or in combination.

FIG. 11 depicts the in vitro treatment of MIA PaCa-2 cells with a chloroquine or CPI-613 alone or in combination.

FIG. 12 depicts the in vitro treatment of CoLo 205 and LoVo cells with a combination of chloroquine and CPI-613.

FIG. 13 depicts the in vitro treatment of SW620 and HT-29 cells with a combination of chloroquine and CPI-613.

FIG. 14 depicts the in vitro treatment of H460 cells with a combination of chloroquine and CPI-613.

FIG. 15 depicts the fluorescence intensity of DALGreen in HS-MM cells treated by vehicle alone (A), 1 μg/mL CPI-613 (B), or 1 μg/mL CPI-613 plus 10 μg/mL chloroquine (C) (Scale bar: 20 μm), or by vehicle alone (D) or CPI-613 plus chloroquine (E) analyzed with a Guava EasyCyte cell analyzer.

FIG. 16 A-F depicts the fluorescence intensity evaluated by a cell analyzer of fluorescein isothiocyanate (FITC)-conjugated Annexin V and propidium iodide (PI) in HS-MM cells untreated (A), treated with 10 μg/mL CPI-613 (B), treated with 10 μg/mL chloroquine (F), or treated with a combination of 10 μg/mL chloroquine and CPI-613 (100 ng/mL, C) (1 μg/mL, D) (10 μg/mL, E). G depicts the fluorescence intensity evaluated by a confocal fluorescence microscope of FITC-conjugated Annexin V and PI in HS-MM cells treated or untreated with 1 μg/mL CPI-613, 10 μg/mL chloroquine, or both. H depicts the fluorescence intensity evaluated by a cell analyzer of FITC-conjugated Annexin V and PI in HS-MM cells untreated (Mock) or treated with a combination of 1 μg/mL CPI-613 and 10 μg/mL chloroquine in the absence or presence of necrostatin-1 (5 μM, 10 μM, or 50 μM). I depicts the fluorescence intensity evaluated by a confocal fluorescence microscope of FITC-conjugated Annexin V and PI in HS-MM cells untreated (Mock) or treated with a combination of 1 μg/mL CPI-613 and 10 μg/mL chloroquine in the presence of necrostatin-1 (5 μM, 10 μM, or 50 μM).

FIG. 17 depicts the efficacy of a combination of CPI-613 and chloroquine in an orthotropic xenoplanted model of clear cell sarcoma (CCS) in SCID-beige mice. 17A depicts tumor volume at the injection site following IP injection of vehicle only (Mock), CPI-613, chloroquine, or a combination of CPI-613 and chloroquine. Arrows indicate day of injection. Data are expressed as means±SD (n=5). 17B depicts total weights of collected, disseminated mesenteric tumors after seven days from the last CPI-613 and chloroquine injections. Data are expressed as means±SD (n=5).

FIG. 18 depicts the efficacy of a combination of CPI-613 and chloroquine in an orthotropic xenoplanted model of clear cell sarcoma (CCS) in male SCID-beige mice. 18A depicts tumor volume at the injection site following IP injection of vehicle only (Mock) or a combination of CPI-613 and chloroquine (CPI-613). Arrows indicate day of injection (twice weekly). 18B depicts total weights of collected, disseminated mesenteric tumors after seven days from the last CPI-613 and chloroquine injections. Data are expressed as means±SD (n=5). (P<0.01). 18C depicts representative mice. The white arrow indicates the distant metastasis.

FIG. 19 depicts the anti-tumor efficacy of oral 6,8-bis-benzylthio-octanoic acid in human non-small cell lung cancer xenografts in mice.

FIG. 20 depicts the anti-tumor efficacy of oral 6,8-bis-benzylthio-octanoic acid in human pancreatic cancer xenografts in mice.

FIG. 21 presents X-ray powder diffraction patterns of solid amorphous dispersion formulations of 6,8-bis-benzylthio-octanoic acid with either Eudragit L100 or hydroxypropyl methylcellulose acetate succinate (HPMCAS-M) (top and middle diffraction patterns, respectively), and crystalline 6,8-bis-benzylthio-octanoic acid (bottom diffraction pattern).

### DETAILED DESCRIPTION

The invention provides methods and compositions for treating cancer in a human patient in need thereof by administering to the patient a therapeutically effective amount of 6,8-bis-benzylthio-octanoic acid and an autophagy inhibitor.

The practice of the present invention employs, unless otherwise indicated, conventional techniques of medicinal chemistry, pharmacology, and biochemistry. Various aspects of the invention are set forth below in sections; however, aspects of the invention described in one particular section are not to be limited to any particular section.

### I. DEFINITIONS

To facilitate an understanding of the present invention, a number of terms and phrases are defined below.

The terms “a,” “an” and “the” as used herein mean “one or more” and include the plural unless the context is inappropriate

The term “6,8-bis-benzylthio-octanoic acid” refers to the compound known as devimistat or CPI-613, having the chemical structure

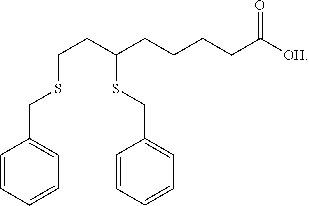 [SMILES: O=C(O)CCCCC(CCSCc1ccccc1)SCc1ccccc1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to a human being in need of cancer treatment.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

“Therapeutically effective amount” refers to an amount of a compound sufficient to inhibit, halt, or cause an improvement in a disorder or condition being treated in a particular patient or patient population. For example, a therapeutically effective amount can be an amount of drug sufficient to slow the progression of a disease, or to prevent or delay its recurrence, such as maintenance treatment to prevent or delay relapse. A therapeutically effective amount can be determined experimentally in a laboratory or clinical setting, or may be the amount required by the guidelines of the United States Food and Drug Administration, or equivalent foreign agency, for the particular disease and patient being treated. It should be appreciated that determination of proper dosage forms, dosage amounts, and routes of administration is within the level of ordinary skill in the pharmaceutical and medical arts.

As used herein, the term “pharmaceutical composition” refers to the combination of an active agent with an excipient, inert or active, making the composition suitable for administration to a human.

The phrase “pharmaceutically acceptable” is employed herein to refer to those compounds, materials, compositions, and/or dosage forms which are, within the scope of sound judgment, suitable for use in contact with the tissues of human beings with acceptable toxicity, irritation, allergic response, and other problems or complications commensurate with a reasonable benefit/risk ratio.

As used herein, the term “pharmaceutically acceptable excipient” refers to any of the standard pharmaceutical excipients suitable for use in humans. For examples of excipients, see e.g., Martin, Remington's Pharmaceutical Sciences, 15th Ed., Mack Publ. Co., Easton, Pa. [1975].

As used herein, the term “pharmaceutically acceptable salt” refers to any salt (e.g., acid or base) of a compound of the present invention which is suitable for administration to a human. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Further examples of salts include salts made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, UIPAC, Wiley-VCH, P. H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified. Further, if a variable is not accompanied by a definition, then the previous definition of the variable controls.

### II. THERAPEUTIC APPLICATIONS

The invention provides methods and compositions for treating cancer in a human patient in need thereof, comprising the step of administering to the patient a therapeutically effective amount of 6,8-bis-benzylthio-octanoic acid and an autophagy inhibitor.

The inventors have discovered that when exposed to CPI-613, cancer cells display increased autophagy. Without wishing to be bound by theory, the inventors hypothesize that when CPI-613 interferes with the altered metabolic pathways of cancer cells, the cells begin to starve and respond by using autophagy to help supply their metabolic needs. Including an autophagy inhibitor in the treatment regimen inhibits the cancer cells from using autophagy to counter the effect of CPI-613, significantly improving efficacy.

### Type of Cancer

In certain embodiments, the cancer is associated with altered energy metabolism. In certain embodiments, the cancer displays increased autophagy when contacted with CPI-613. As used herein, the term “cancer” is intended to include myelodysplastic syndromes, and in certain embodiments of the present invention the cancer is a myelodysplastic syndrome. In certain embodiments, the cancer is high risk myelodysplastic syndrome (MDS). In certain embodiments, the cancer is high risk MDS in patients who have failed to respond, progressed, or relapsed while on hypomethylating therapy.

The method may be further characterized according to the severity or type of cancer. In certain embodiments, the cancer is Stage I or early stage cancer, in which the cancer is small and only in one area. In certain embodiments, the cancer is Stage II or III, in which the cancer is larger and has grown into nearby tissues or lymph nodes. In certain embodiments, the cancer Stage IV or advanced or metastatic, in which the cancer has spread to other parts of the body.

In certain embodiments, the cancer is Stage I lymphoma, in which the cancer is found in one lymph node region or the cancer has invaded one extralymphatic organ or site but not any lymph node regions. In certain embodiments, the cancer is Stage II lymphoma, in which the cancer is found in two or more lymph node regions on the same side of the diaphragm or the cancer involves one organ and its regional lymph nodes, with or without cancer in other lymph node regions on the same side of the diaphragm. In certain embodiments, the cancer is Stage III lymphoma, in which there is cancer in lymph nodes on both sides of the diaphragm. In certain embodiments, the cancer is Stage IV lymphoma, in which the cancer has spread one or more organs beyond the lymph nodes.

In certain embodiments, the cancer is progressive or refractory. In certain embodiments, the cancer is a metastatic. In certain embodiments, the cancer is recurrent or relapsed. In certain embodiments, the cancer is relapsed or refractory. In certain embodiments, the cancer is previously untreated. In certain embodiments, the cancer is previously untreated with systemic therapies. In certain embodiments, the cancer is previously untreated with systemic therapies or local treatment with chemoradiation. In certain embodiments, the patient has not received hematopoietic cell transplant. In certain embodiments, the patient has received hematopoietic cell transplant.

In certain embodiments, the cancer is a lymphoma. In certain embodiments, the cancer is a T-cell lymphoma. In certain embodiments, the cancer is a B-cell lymphoma. In certain embodiments, the cancer is mantle cell lymphoma. In certain embodiments, the cancer is a leukemia. In certain embodiments, the cancer is an acute myeloid leukemia. In certain embodiments, the cancer is chronic myeloid leukemia. In certain embodiments, the cancer is acute lymphoblastic leukemia. In certain embodiments, the cancer is a carcinoma. In certain embodiments, the cancer is a sarcoma. In certain embodiments, the cancer is a myeloma. In certain embodiments, the cancer is a clear cell cancer. In certain embodiments, the cancer is a clear cell sarcoma. In certain embodiments, the cancer is a clear cell carcinoma. In certain embodiments, the cancer is a brain or spinal cord cancer. In certain embodiments, the cancer is a melanoma. In certain embodiments, the cancer is a blastoma. In certain embodiments, the cancer is a germ cell tumor. In certain embodiments, the cancer is a cancer of the pancreas. In certain embodiments, the cancer is a metastatic pancreatic cancer. In certain embodiments, the cancer is a locally advanced pancreatic cancer. In certain embodiments, the cancer is a histologically or cytologically documented and measurable locally advanced pancreatic adenocarcinoma. In certain embodiments, the cancer is a histologically or cytologically documented and measurable metastatic pancreatic adenocarcinoma. In certain embodiments, the cancer is a histologically or cytologically documented and measurable locally advanced pancreatic adenocarcinoma that is previously untreated. In certain embodiments, the cancer is a histologically or cytologically documented and measurable metastatic pancreatic adenocarcinoma that is previously untreated. In certain embodiments, the cancer is a histologically or cytologically documented and measurable locally advanced pancreatic adenocarcinoma that is previously untreated with systemic therapies. In certain embodiments, the cancer is a histologically or cytologically documented and measurable metastatic pancreatic adenocarcinoma that is previously untreated with systemic therapies. In certain embodiments, the cancer is a histologically or cytologically documented and measurable locally advanced pancreatic adenocarcinoma that is previously untreated with systemic therapies or local treatment with chemoradiation. In certain embodiments, the cancer is a histologically or cytologically documented and measurable metastatic pancreatic adenocarcinoma that is previously untreated with systemic therapies or local treatment with chemoradiation. In certain embodiments, the cancer is a locally advanced pancreatic adenocarcinoma. In certain embodiments, the cancer is a metastatic pancreatic adenocarcinoma. In certain embodiments, the cancer is a locally advanced pancreatic adenocarcinoma that is previously untreated. In certain embodiments, the cancer is a metastatic pancreatic adenocarcinoma that is previously untreated. In certain embodiments, the cancer is a locally advanced pancreatic adenocarcinoma that is previously untreated with systemic therapies. In certain embodiments, the cancer is a metastatic pancreatic adenocarcinoma that is previously untreated with systemic therapies. In certain embodiments, the cancer is a locally advanced pancreatic adenocarcinoma that is previously untreated with systemic therapies or local treatment with chemoradiation. In certain embodiments, the cancer is a pancreatic adenocarcinoma that is previously untreated with systemic therapies or local treatment with chemoradiation.

In certain embodiments, the cancer is a cancer of the prostate. In certain embodiments, the cancer is a castration resistant prostate cancer. In certain embodiments, the cancer is a cancer of the lung. In certain embodiment, the cancer is non-small cell lung cancer. In certain embodiments, the cancer is a cancer of the colon. In certain embodiments, the cancer is a cancer of the rectum. In certain embodiments, the cancer is a colorectal cancer. In certain embodiments, the cancer is a cancer of the cervix. In certain embodiments, the cancer is a neuroendocrine tumor. In certain embodiments, the cancer is a gastroenteropancreatic neuroendocrine tumor. In certain embodiments, the cancer is a cancer of the liver. In certain embodiments, the cancer is a cancer of the uterus. In certain embodiments, the cancer is a cancer of the cervix. In certain embodiments, the cancer is a cancer of the bladder. In certain embodiments, the cancer is a cancer of the kidney. In certain embodiments, the cancer is a cancer of the breast. In certain embodiments, the cancer is a cancer of the ovary.

In certain embodiments, the cancer is Burkitt's Lymphoma. In certain embodiments, the cancer is relapsed or refractory Burkitt's Lymphoma. In certain embodiments, the cancer is relapsed or refractory Burkitt's Lymphoma in which the patient has failed at least one previous line of therapy. In certain embodiments, the cancer is relapsed or refractory Burkitt's Lymphoma in which the patient has failed prior bone marrow transplant. In certain embodiments, the cancer is double hit diffuse large B cell lymphoma. In certain embodiments, the cancer is high-grade B cell lymphoma with rearrangements of MYC and BCL2 and/or BCL6 (DHL/THL). In certain embodiments, the cancer is Hodgkin lymphoma. In certain embodiments, the cancer is non-Hodgkin lymphoma. In certain embodiments, the cancer is T-cell non-Hodgkin lymphoma. In certain embodiments, the cancer is relapsed or refractory Hodgkin lymphoma. In certain embodiments, the cancer is relapsed or refractory non-Hodgkin lymphoma. In certain embodiments, the cancer is relapsed or refractory T-cell non-Hodgkin lymphoma. In certain embodiments, the cancer is Hodgkin lymphoma in which the patient has not received hematopoietic cell transplant. In certain embodiments, the cancer is Hodgkin lymphoma in which the patient has received hematopoietic cell transplant. In certain embodiments, the cancer is non-Hodgkin lymphoma in which the patient has not received hematopoietic cell transplant. In certain embodiments, the cancer is non-Hodgkin lymphoma in which the patient has received hematopoietic cell transplant. In certain embodiments, the cancer is T-cell non-Hodgkin lymphoma in which the patient has not received hematopoietic cell transplant. In certain embodiments, the cancer is T-cell non-Hodgkin lymphoma in which the patient has received hematopoietic cell transplant. In certain embodiments, the cancer is relapsed or refractory Hodgkin lymphoma in which the patient has not received hematopoietic cell transplant. In certain embodiments, the cancer is relapsed or refractory Hodgkin lymphoma in which the patient has received hematopoietic cell transplant. In certain embodiments, the cancer is relapsed or refractory non-Hodgkin lymphoma in which the patient has not received hematopoietic cell transplant. In certain embodiments, the cancer is relapsed or refractory Hodgkin lymphoma in which the patient has or has not received hematopoietic cell transplant. In certain embodiments, the cancer is relapsed or refractory Hodgkin lymphoma in which the patient has failed brentuximab vedotin and a PD-1 inhibitor. In certain embodiments, the cancer is relapsed or refractory Hodgkin lymphoma in which the patient has failed brentuximab vedotin and a PD-1 inhibitor and has received hematopoietic cell transplant. In certain embodiments, the cancer is relapsed or refractory Hodgkin lymphoma in which the patient has failed brentuximab vedotin and a PD-1 inhibitor and has not received hematopoietic cell transplant. In certain embodiments, the cancer is relapsed or refractory non-Hodgkin lymphoma in which the patient has received hematopoietic cell transplant. In certain embodiments, the cancer is relapsed or refractory T-cell non-Hodgkin lymphoma in which the patient has not received hematopoietic cell transplant. In certain embodiments, the cancer is relapsed or refractory T-cell non-Hodgkin lymphoma in which the patient has received hematopoietic cell transplant. In certain embodiments, the cancer is relapsed or refractory T-cell non-Hodgkin lymphoma in which the patient has or has not received hematopoietic cell transplant.

### General Aspects of Administering a Therapeutic Agent to a Patient

Generally, the therapeutic agent—i.e., 6,8-bis-benzylthio-octanoic acid and autophagy inhibitor—is delivered to the patient in a therapeutically effective amount, sufficient to treat cancer. The treatment may involve one or several administrations on one or more days, and the dosage may be adjusted by the individual practitioner to achieve a desired effect. Preferably, the dosage amount of each agent should be sufficient to interact primarily with disease cells, leaving normal cells comparatively unharmed.

The dosage amount may be administered in a single dose or in the form of individual divided doses, such as one, two, three, or four times per day. In certain embodiments, the daily dosage amount is administered in a single dose. In the event that the response in a patient is insufficient at a certain dose, higher or more frequent doses may be employed to the extent of patient tolerance.

For the present combination therapy, each agent may be administered in a particular order and/or on the same or different days according to a treatment cycle. For example, a dose of 6,8-bis-benzylthio-octanoic acid may be administered to the patient prior to administering an autophagy inhibitor, such as immediately prior, earlier in the day, or on an earlier day in a treatment cycle. In certain embodiments, the active agents may be administered on the same day of a treatment cycle, for example being co-administered simultaneously or one right after the other. In certain embodiments, a dose of an autophagy inhibitor is administered to the patient prior to administering the 6,8-bis-benzylthio-octanoic acid, such as immediately prior, earlier in the day, or on an earlier day in a treatment cycle. In certain embodiments, treatment cycles may be repeated one or more times in order to maximize benefit to the patient.

### 6,8-Bis-Benzylthio-Octanoic Acid

The 6,8-bis-benzylthio-octanoic acid may be administered in any suitable form, including as a solid or liquid, a free acid or salt. The 6,8-bis-benzylthio-octanoic acid may be crystalline, amorphous, or dissolved in solution. In certain embodiments, the 6,8-bis-benzylthio-octanoic acid is administered to the patient as a salt or ion pair. In certain embodiments, the 6,8-bis-benzylthio-octanoic acid is administered to the patient as a salt or ion pair with triethanolamine. Exemplary ion pairing agents that may be used include, for example, a tertiary amine (such as triethylamine or triethanolamine), other amines such as diethylamine, diethanolamine, monoethanolamine, mefenamic acid and tromethamine, and combinations thereof. In certain embodiments, the ion pairing agent is an organic Bronsted base. In certain other embodiments, the ion pairing agent is an amine compound. In yet other embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom. In certain embodiments, the therapeutic agent is a salt of 6,8-bis-benzylthio-octanoic acid with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

Additional exemplary ion pairing agents include, for example, polyethyleneimine, polyglutamic acid, ammonia, L-arginine, benethamine benzathine, betaine, calcium hydroxide, choline, deanol, diethanolamine (2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain other embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain other embodiments, the ion pairing agent is an alkali metal hydroxide or alkaline earth metal hydroxide, such as, for example, cesium hydroxide.

In certain embodiments, the 6,8-bis-benzylthio-octanoic acid has a purity of at least about 50% (w/w). In certain embodiments, the 6,8-bis-benzylthio-octanoic acid has a purity of at least about 60% (w/w). In certain embodiments, the 6,8-bis-benzylthio-octanoic acid has a purity of at least about 70% (w/w). In certain embodiments, the 6,8-bis-benzylthio-octanoic acid has a purity of at least about 80% (w/w). In certain embodiments, the 6,8-bis-benzylthio-octanoic acid has a purity of at least about 90% (w/w). In certain embodiments, the 6,8-bis-benzylthio-octanoic acid has a purity of at least about 95% (w/w). In certain embodiments, the 6,8-bis-benzylthio-octanoic acid has a purity of at least about 96% (w/w). In certain embodiments, the 6,8-bis-benzylthio-octanoic acid has a purity of at least about 97% (w/w). In certain embodiments, the 6,8-bis-benzylthio-octanoic acid has a purity of at least about 98% (w/w). In certain embodiments, the 6,8-bis-benzylthio-octanoic acid has a purity of at least about 99% (w/w).

### Autophagy Inhibitor

The one or more autophagy inhibitors may be administered in any suitable form, including as a solid or liquid, a free acid or salt. The autophagy inhibitor may be crystalline, amorphous, or dissolved in solution. In certain embodiments, the autophagy inhibitor is administered to the patient as a salt or ion pair. When the autophagy inhibitor is a basic compound, such as chloroquine or hydroxychloroquine, it may be administered as an ion pair with an inorganic or organic acid. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. In certain embodiments, the therapeutic agent is a salt of an autophagy inhibitor with an ion pairing agent selected with guidance from Berge et al., “Pharmaceutical Salts,” J. of Pharmaceutical Science, 1977; 66:1-19 or Handbook of Pharmaceutical Salts Properties, Selection and Use, IUPAC, Wiley-VCH, P. H. Stahl, ed., the entire disclosures of which are incorporated by reference herein. Ion pairing agents of particular note in the latter include, without limitation, those listed in Table 5, p. 342.

Any suitable autophagy inhibitor may be used. In certain embodiments, the autophagy inhibitor is chosen from a 4-aminoquinoline, 3-methyladenine (3-MA, CAS #5142-23-4), MHY1485 (CAS #326914-06-1SP600125), 3-methyl-6-(3-methylpiperidin-1-yl)-3H-purine, 6-Chloro-N-(1-ethylpiperidin-4-yl)-1,2,3,4-tetrahydroacridin-9-amine, 4-(((1-(2-Fluorophenyl)cyclopentyl)-amino)methyl)-2-((4-methylpiperazin-1-yl)methyl)phenol, 6-fluoro-N-[4-fluorobenzyl]quinazolin-4-amine, N-acetyl-L-cysteine, L-asparagine, N2,N4-dibenzylquinazoline-2,4-diamine, (2S,3S)-trans-Epoxysuccinyl-L-leucylamido-3-methylbutane ethyl ester, N-[6-(4-chlorophenoxy)hexyl]-N′-cyano-N″-4-pyridinyl-guanidine, leupeptin, 2-(4-Morpholinyl)-8-phenyl-1(4H)-benzopyran-4-one, 4,6-Di-4-morpholinyl-N-(4-nitrophenyl)-1,3,5-triazin-2-amine, pepstatin A, 2-((5-Bromo-2-((3,4,5-trimethoxyphenyl)amino)pyrimidin-4-yl)oxy)-N-methylbenzamide, 6-Fluoro-N-[(4-fluorophenyl)methyl]-4-quinazolinamine, thapsigargin, amodiaquine, artemisinin, mefloquine, primaquine, piperaquine, quinacrine, U0126, 3-methyladenine, bafilomycin A1, chloroquine, hydroxychloroquine, verteporfin, LY294002, SB202190, SB203580, SC79, and wortmannin. In certain embodiments, the autophagy inhibitor is chosen from chloroquine, hydroxychloroquine, and verteporfin. In certain embodiments, the autophagy inhibitor is chosen from hydroxychloroquine and verteporfin. In certain embodiments, the autophagy inhibitor is a 4-aminoquinoline. In certain embodiments, the autophagy inhibitor is chloroquine. In certain embodiments, the autophagy inhibitor is chloroquine phosphate. In certain embodiments, the autophagy inhibitor is chloroquine sulfate. In certain embodiments, the autophagy inhibitor is chloroquine hydrochloride. In certain embodiments, the autophagy inhibitor is hydroxychloroquine. In certain embodiments, the autophagy inhibitor is hydroxychloroquine sulfate. In certain embodiments, the autophagy inhibitor is verteporfin.

The autophagy inhibitor may inhibit any suitable type of autophagy (e.g., macroautophagy, microautophagy, chaperone-mediated autophagy, mitophagy, or lipophagy), and may do so by any suitable mechanism (e.g., by impacting formation of an autophagosome or its cargo). In certain embodiments, the autophagy inhibitor inhibits macroautophagy or mitophagy. In certain embodiments, the autophagy inhibitor inhibits macroautophagy. In certain embodiments, the autophagy inhibitor inhibits mitophagy. In certain embodiments, the mitophagy inhibitor is Mdivi-1. In certain embodiments, the mitophagy inhibitor is cyclosporine A. In certain embodiments, the autophagy inhibitor inhibits, microautophagy. In certain embodiments, the autophagy inhibitor inhibits chaperone-mediated autophagy. In certain embodiments, the autophagy inhibitor inhibits lipophagy.

### Route of Administration

The 6,8-bis-benzylthio-octanoic acid and autophagy inhibitor may be administered to the patient by any suitable route. For example, in certain embodiments, the 6,8-bis-benzylthio-octanoic acid and/or autophagy inhibitor is administered orally to the patient. In certain embodiments, the 6,8-bis-benzylthio-octanoic acid and autophagy inhibitor are administered orally to the patient. In certain embodiments, the 6,8-bis-benzylthio-octanoic acid is administered orally to the patient. In certain embodiments, the autophagy inhibitor is administered orally to the patient. In certain embodiments, the 6,8-bis-benzylthio-octanoic acid and/or autophagy inhibitor is administered subcutaneously to the patient. In certain embodiments, the 6,8-bis-benzylthio-octanoic acid and/or autophagy inhibitor is administered intravenously to the patient. In certain embodiments, the 6,8-bis-benzylthio-octanoic acid is administered as an IV infusion over two hours. In certain embodiments, the 6,8-bis-benzylthio-octanoic acid is administered as an IV infusion over two hours via a central venous catheter.

An advantage of oral dosing of the 6,8-bis-benzylthio-octanoic acid is that it permits substantially increased dosing flexibility as compared to IV. In the prior art, 6,8-bis-benzylthio-octanoic acid is formulated as a 50 mg/mL solution in 1 M (150 mg/mL) aqueous triethanolamine, which is diluted from 50 mg/mL to as low as 4 mg/mL (e.g., 12.5 mg/mL) with sterile 5% dextrose for injection (D5W) prior to administration as an IV infusion over 30-120 minutes via a central venous catheter. Such an infusion is inconvenient for patients and effectively precludes regimens involving frequent and/or prolonged dosing. Since the half-life of 6,8-bis-benzylthio-octanoic acid after IV dosing is only about 1-2 hours (Pardee, T. S. et al., Clin Cancer Res. 2014, 20, 5255-64), more frequent and/or prolonged dosing could advantageously be used to increase the patient's exposure to the drug.

For example, a possible IV schedule for the treatment of high risk MDS involves administering hydroxychloroquine (600 mg to 1,200 mg) orally on days 1-5 of a 28 day cycle, followed each day by 6,8-bis-benzylthio-octanoic acid (2,000 mg/m²) IV. If administered orally, the practitioner would have more flexibility with respect to the 6,8-bis-benzylthio-octanoic acid dose and schedule. The 6,8-bis-benzylthio-octanoic acid could be orally administered in a single daily dose on days 1-5 of a 28 day cycle as in the IV schedule. Alternatively, the 6,8-bis-benzylthio-octanoic acid could be administered in two or more (e.g., three, four, or five) divided doses. The single or divided doses could be administered on days 1-5 of the 28 day cycle or on fewer and/or additional days of the cycle, up to and including every day.

Another advantage of oral dosing is that it makes maintenance therapy feasible. For example, a patient who is treated successfully with first line therapy—with or without 6,8-bis-benzylthio-octanoic acid—and whose cancer is in partial or complete remission, may be treated orally with 6,8-bis-benzylthio-octanoic acid and an autophagy inhibitor (e.g., hydroxychloroquine) on a chronic basis in order to delay or prevent recurrence. The maintenance treatment may involve, for example, one, two, three, four, or five doses per day of the 6,8-bis-benzylthio-octanoic acid and autophagy inhibitor on a regular basis, such as daily or weekly. In certain embodiments, the maintenance therapy is for the treatment of pancreatic cancer.

### Pharmaceutical Composition

Any suitable pharmaceutical composition may be used to administer the 6,8-bis-benzylthio-octanoic acid and the autophagy inhibitor to the patient. The therapeutic agents may be administered together in the same pharmaceutical composition (e.g., fixed dose combination) or separately in different pharmaceutical compositions. There is a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000). In certain embodiments, one or more of the therapeutic agents is administered in a pharmaceutical composition that is a dry oral dosage form. In certain embodiments, the pharmaceutical composition is an oral dosage form chosen from tablet, pill, capsule, caplet, powder, granule, solution, suspension, and gel. Oral dosage forms may include pharmaceutically acceptable excipients, such as carriers, diluents, stabilizers, plasticizers, binders, glidants, disintegrants, bulking agents, lubricants, plasticizers, colorants, film formers, flavoring agents, preservatives, dosing vehicles, and any combination of any of the foregoing.

The pharmaceutical composition will generally include at least one inert excipient. Excipients include pharmaceutically compatible binding agents, lubricants, wetting agents, disintegrants, and the like. Tablets, pills, capsules, troches and the like can contain any of the following excipients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain a liquid excipient such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. In certain embodiments, the pharmaceutical composition comprises an excipient in an amount of about 5% to about 99%, such as about 10% to about 85%, by weight of the composition, with the therapeutic agent comprising the remainder. In certain embodiments, pharmaceutically acceptable excipients comprise about 20% to about 80% of the total weight of the composition. In certain embodiments, the pharmaceutical composition comprises the therapeutic agent in an amount of at least about 40% by weight of the composition, with one or more excipients comprising the remainder. In certain embodiments, the pharmaceutical composition comprises the therapeutic agent in an amount of at least about 50% by weight of the composition. In certain embodiments, the pharmaceutical composition comprises the therapeutic agent in an amount of at least about 60% by weight of the composition. In certain embodiments, the pharmaceutical composition comprises the therapeutic agent in an amount of at least about 70% by weight of the composition. In certain embodiments, the pharmaceutical composition comprises the therapeutic agent in an amount of at least about 80% by weight of the composition. In certain embodiments, the pharmaceutical composition comprises the therapeutic agent in an amount of at least about 90% by weight of the composition.

Diluents for solid (e.g., oral) compositions include, but are not limited to, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit), potassium chloride, powdered cellulose, sodium chloride, sorbitol and talc.

Binders for solid (e.g., oral) pharmaceutical compositions include, but are not limited to, acacia, tragacanth, sucrose, glucose, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch. In certain embodiments, the pharmaceutical composition comprises a binder in an amount of about 0.5% to about 25%, such as about 0.75% to about 15%, by weight of the composition. In certain embodiments, the pharmaceutical composition comprises a binder in an amount of about 1% to about 10% by weight of the composition.

The dissolution rate of a compacted solid pharmaceutical composition in a patient's stomach may be increased by the addition of a disintegrant to the composition. Disintegrants include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, powdered cellulose, pregelatinized starch, sodium alginate, sodium starch glycolate (e.g. EXPLOTAB®) and starch. In certain embodiments, the pharmaceutical composition comprises a disintegrant in an amount of about 0.2% to about 30%, such as about 0.2% to about 10%, by weight of the composition. In certain embodiments, the pharmaceutical composition comprises a disintegrant in an amount of about 0.2% to about 5% by weight of the composition.

The pharmaceutical composition optionally comprises one or more pharmaceutically acceptable wetting agents. Such wetting agents are preferably selected to maintain the API in close association with water, a condition that is believed to improve bioavailability of the composition. Non-limiting examples of surfactants that can be used as wetting agents include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene, caprylic/capric mono- and diglycerides (e.g., Labrasol™ of Gattefosse), polyoxyethylene castor oil and polyoxyethylene hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. In certain embodiments, the pharmaceutical composition comprises a wetting agent in an amount of about 0.25% to about 15%, such as about 0.4% to about 10%, by weight of the composition. In certain embodiments, the pharmaceutical composition comprises a wetting agent in an amount of about 0.5% to about 5% by weight of the composition. In certain embodiments, the pharmaceutical composition comprises a wetting agent that is an anionic surfactant. In certain embodiments, the pharmaceutical composition comprises sodium lauryl sulfate as a wetting agent. In certain embodiments, the pharmaceutical composition comprises sodium lauryl sulfate in an amount of about 0.25% to about 7%, such as about 0.4% to about 4%, by weight of the composition. In certain embodiments, the pharmaceutical composition comprises sodium lauryl sulfate in an amount of about 0.5% to about 2% by weight of the composition.

Lubricants (e.g., anti-adherents or glidants) can be added to improve the flow properties of solid compositions and/or to reduce friction between the composition and equipment during compression of tablet formulations. Excipients that may function as lubricants include, but are not limited to, colloidal silicon dioxide, magnesium trisilicate, powdered cellulose, starch, talc and tribasic calcium phosphate. Suitable lubricants further include glyceryl behapate (e.g., Compritol™ 888 of Gattefosse); stearic acid and salts thereof, including magnesium, calcium and sodium stearates; zinc stearate; glyceryl monostearate; glyceryl palmitostearate; hydrogenated castor oil; hydrogenated vegetable oils (e.g., Sterotex™ of Abitec); waxes; boric acid; sodium benzoate; sodium acetate; sodium stearyl fumarate; sodium fumarate; sodium chloride; DL-leucine; PEG (e.g., Carbowax™ 4000 and Carbowax™ 6000 of the Dow Chemical Company); sodium oleate; sodium lauryl sulfate; and magnesium lauryl sulfate. In certain embodiments, the pharmaceutical compositions comprises a lubricant in an amount of about 0.1% to about 10%, such as about 0.2% to about 8%, by weight of the composition. In certain embodiments, the pharmaceutical composition comprises a lubricant in an amount of about 0.25% to about 5% by weight of the composition. In certain embodiments, the pharmaceutical composition comprises magnesium stearate as a lubricant. In certain embodiments, the pharmaceutical composition comprises colloidal silicon dioxide. In certain embodiments, the pharmaceutical composition comprises talc. In certain embodiments, the composition comprises magnesium stearate or talc in an amount of about 0.5% to about 2% by weight of the composition.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level. The formulations of the invention may be buffered by the addition of suitable buffering agents.

In certain embodiments of the present invention, the therapeutic agent may be formulated as a pharmaceutically-acceptable oil; liposome; oil-water or lipid-oil-water emulsion or nanoemulsion; or liquid. To facilitate such formulations, the therapeutic agent may be combined with a pharmaceutically-acceptable excipient therefor.

As described in detail below, the pharmaceutical compositions may be specially formulated for administration in solid or liquid form, including those adapted for parenteral administration, for example, by subcutaneous, intramuscular, intravenous or epidural injection as, for example, a sterile solution or suspension, or sustained-release formulation.

Further examples of pharmaceutical formulations of 6,8-bis-benzylthio-octanoic acid are described in U.S. Pat. No. 8,263,653, the entire disclosure of which is incorporated by reference herein.

Methods of preparing pharmaceutical formulations or pharmaceutical compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

In certain embodiments, the pharmaceutical composition comprising the first therapeutic agent is a spray-dried dispersion. In certain embodiments, the pharmaceutical composition comprising the first therapeutic agent is a spray-dried dispersion comprising at least one polymer chosen from polyacrylate, polymethacrylate, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition comprising the first therapeutic agent is a spray-dried dispersion comprising at least one polymer chosen from Eudragit L100, poly(vinylpyrrolidone), hydroxypropyl methyl cellulose (HPMC), cellulose acetate phthalate (CAP), and hydroxypropyl methylcellulose acetate succinate (HPMCAS-M). In certain embodiments, the pharmaceutical composition comprising the first therapeutic agent is a spray-dried dispersion comprising at least one polymer chosen from Eudragit L100, poly(vinylpyrrolidone) viscosity grade K30 (PVP K30), hydroxypropyl methyl cellulose (HPMC), cellulose acetate phthalate (CAP), and hydroxypropyl methylcellulose acetate succinate (HPMCAS-M). In certain embodiments, the pharmaceutical composition comprising the first therapeutic agent is a spray-dried dispersion comprising at least one polymer chosen from Eudragit L100 and hydroxypropyl methylcellulose acetate succinate (HPMCAS-M). In certain embodiments, the pharmaceutical composition comprising the first therapeutic agent is a spray-dried dispersion comprising Eudragit L100. In certain embodiments, the pharmaceutical composition comprising the first therapeutic agent is a spray-dried dispersion comprising hydroxypropyl methylcellulose acetate succinate (HPMCAS-M).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

In certain embodiments, one or more of the therapeutic agents are administered by intraparenteral administration. In certain other embodiments, one or more of the therapeutic agents are formulated for inhalational, oral, topical, transdermal, nasal, ocular, pulmonary, rectal, transmucosal, intravenous, intramuscular, subcutaneous, intraperitoneal, intrathoracic, intrapleural, intrauterine, intratumoral, or infusion methodologies or administration, or combinations of any thereof, in the form of aerosols, sprays, powders, gels, lotions, creams, suppositories, ointments, and the like. As indicated above, if such a formulation is desired, other additives known in the art may be included to impart the desired consistency and other properties to the formulation.

In certain embodiments, the pharmaceutical composition of the present invention is a unit dose composition. In certain embodiments, the pharmaceutical composition contains about 1 mg to about 5000 mg of the therapeutic agent. In certain embodiments, the pharmaceutical composition contains about 100 mg to about 3000 mg of the therapeutic agent. In certain embodiments, the pharmaceutical composition contains about 200 mg to about 2000 mg of the therapeutic agent. In certain embodiments, the pharmaceutical composition contains about 50 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2500 mg, or 3000 mg of therapeutic agent. In certain embodiments, the pharmaceutical composition contains about 300 mg, 500 mg, 700 mg, or 1000 mg of the therapeutic agent.

In certain embodiments, the pharmaceutical composition of the present invention comprises an emulsion, particle, or gel as described in U.S. Pat. No. 7,220,428. In certain embodiments, the pharmaceutical composition is a solid or liquid formulation having from about 0.1% to about 75% w/w lipids or fatty acid components. In certain embodiments, the formulation contains about 0.1% to about 15% w/v lipids and fatty acid components. In certain embodiments, the fatty acid component comprises saturated or unsaturated C4, C5, C6, C7, C8, C9, C10, C11, or C12 fatty acids and/or salts of such fatty acids. Lipids may include cholesterol and analogs thereof.

In certain embodiments, the pharmaceutical composition of 6,8-bis-benzylthio-octanoic acid comprises triethanolamine and 6,8-bis-benzylthio-octanoic acid in a mole ratio of triethanolamine to 6,8-bis-benzylthio-octanoic acid of about 10:1 to about 1:10. In certain embodiments, the mole ratio of triethanolamine to 6,8-bis-benzylthio-octanoic acid is about 10:1 to about 5:1. In certain embodiments, the mole ratio of triethanolamine to 6,8-bis-benzylthio-octanoic acid is about 8:1. In certain embodiments, the pharmaceutical composition comprises a 50 mg/mL solution of 6,8-bis-benzylthio-octanoic acid in 1M aqueous triethanolamine. In certain embodiments, the pharmaceutical composition comprises a solution of 6,8-bis-benzylthio-octanoic acid in 1M aqueous triethanolamine diluted from 50 mg/mL to as low as 12.5 mg/mL with sterile aqueous 5% dextrose for injection (D5W). In certain embodiments, the pharmaceutical composition comprises a solution of 6,8-bis-benzylthio-octanoic acid in 1M aqueous triethanolamine diluted from 50 mg/mL to about 12.5 mg/mL with sterile aqueous 5% dextrose for injection (D5W).

Several pharmaceutical compositions of autophagy inhibitors are commercially available. In certain embodiments, the pharmaceutical composition of the autophagy inhibitor is an oral tablet comprising chloroquine phosphate in an amount equivalent to 150 mg of the free base. In certain embodiments, the pharmaceutical composition of the autophagy inhibitor is an oral tablet comprising chloroquine phosphate in an amount equivalent to 300 mg of the free base. In certain embodiments, the pharmaceutical composition of the autophagy inhibitor is an oral tablet comprising 200 mg hydroxychloroquine sulfate, equivalent to 155 mg of the free base. In certain embodiments, the pharmaceutical composition of the autophagy inhibitor is an injectable liquid comprising chloroquine hydrochloride in an amount equivalent to 40 mg/mL of the free base.

### Dosing Amounts & Schedules

The 6,8-bis-benzylthio-octanoic acid and autophagy inhibitor may be administered to the patient in any suitable dose according to any suitable schedule. The dose and schedule will vary based on the cancer being treated and can be readily determined by those of ordinary skill in the art in view of the 6,8-bis-benzylthio-octanoic acid and autophagy inhibitor doses and schedules used in the prior art when administered alone or in combination with other agents, as well as the guidance provided herein. In certain embodiments, the dose is the maximum tolerated dose.

In certain embodiments, the 6,8-bis-benzylthio-octanoic acid is administered at a daily dose of about 150 mg/m² to about 3000 mg/m². In certain embodiments, the 6,8-bis-benzylthio-octanoic acid is administered at a daily dose of about 250 mg/m² to about 2500 mg/m². In certain embodiments, the first 6,8-bis-benzylthio-octanoic acid is administered at a daily dose of about 500 mg/m² to about 2000 mg/m². In certain embodiments, the 6,8-bis-benzylthio-octanoic acid is administered at a daily dose of about 150 mg/m². In certain embodiments, the 6,8-bis-benzylthio-octanoic acid is administered at a daily dose of about 200 mg/m². In certain embodiments, the 6,8-bis-benzylthio-octanoic acid is administered at a daily dose of about 250 mg/m². In certain embodiments, the 6,8-bis-benzylthio-octanoic acid is administered at a daily dose of about 300 mg/m². In certain embodiments, the 6,8-bis-benzylthio-octanoic acid is administered at a daily dose of about 350 mg/m². In certain embodiments, the 6,8-bis-benzylthio-octanoic acid is administered at a daily dose of about 400 mg/m². In certain embodiments, the 6,8-bis-benzylthio-octanoic acid is administered at a daily dose of about 450 mg/m². In certain embodiments, the 6,8-bis-benzylthio-octanoic acid is administered at a daily dose of about 500 mg/m². In certain embodiments, the 6,8-bis-benzylthio-octanoic acid is administered at a daily dose of about 600 mg/m². In certain embodiments, the 6,8-bis-benzylthio-octanoic acid is administered at a daily dose of about 700 mg/m². In certain embodiments, the 6,8-bis-benzylthio-octanoic acid is administered at a daily dose of about 800 mg/m². In certain embodiments, the 6,8-bis-benzylthio-octanoic acid is administered at a daily dose of about 900 mg/m². In certain embodiments, the 6,8-bis-benzylthio-octanoic acid is administered at a daily dose of about 1000 mg/m². In certain embodiments, the 6,8-bis-benzylthio-octanoic acid is administered at a daily dose of about 1100 mg/m². In certain embodiments, the 6,8-bis-benzylthio-octanoic acid is administered at a daily dose of about 1200 mg/m². In certain embodiments, the 6,8-bis-benzylthio-octanoic acid is administered at a daily dose of about 1300 mg/m². In certain embodiments, the 6,8-bis-benzylthio-octanoic acid is administered at a daily dose of about 1400 mg/m². In certain embodiments, the 6,8-bis-benzylthio-octanoic acid is administered at a daily dose of about 1500 mg/m². In certain embodiments, the 6,8-bis-benzylthio-octanoic acid is administered at a daily dose of about 1600 mg/m². In certain embodiments, the 6,8-bis-benzylthio-octanoic acid is administered at a daily dose of about 1700 mg/m². In certain embodiments, the 6,8-bis-benzylthio-octanoic acid is administered at a daily dose of about 1800 mg/m². In certain embodiments, the 6,8-bis-benzylthio-octanoic acid is administered at a daily dose of about 1900 mg/m². In certain embodiments, the 6,8-bis-benzylthio-octanoic acid is administered at a daily dose of about 2000 mg/m². In certain embodiments, the 6,8-bis-benzylthio-octanoic acid is administered at a daily dose of about 2500 mg/m². In certain embodiments, the 6,8-bis-benzylthio-octanoic acid is administered at a daily dose of about 3000 mg/m².

In certain embodiments, the 6,8-bis-benzylthio-octanoic acid is administered at a daily dose of about 1 mg to about 10,000 mg. In certain embodiments, the 6,8-bis-benzylthio-octanoic acid is administered at a daily dose of about 10 mg to about 7,500 mg. In certain embodiments, the 6,8-bis-benzylthio-octanoic acid is administered at a daily dose of about 100 mg to about 5,000 mg. In certain embodiments, the 6,8-bis-benzylthio-octanoic acid is administered at a daily dose of about 200 mg to about 4,000 mg. In certain embodiments, the 6,8-bis-benzylthio-octanoic acid is administered at a daily dose of about 300 mg to about 3,000 mg. In certain embodiments, the 6,8-bis-benzylthio-octanoic acid is administered at a daily dose of about 400 mg to about 2,500 mg. In certain embodiments, the 6,8-bis-benzylthio-octanoic acid is administered at a daily dose of about 500 mg to about 2,000 mg. In certain embodiments, the 6,8-bis-benzylthio-octanoic acid is administered at a daily dose of about 100 mg. In certain embodiments, the 6,8-bis-benzylthio-octanoic acid is administered at a daily dose of about 200 mg. In certain embodiments, the 6,8-bis-benzylthio-octanoic acid is administered at a daily dose of about 300 mg. In certain embodiments, the 6,8-bis-benzylthio-octanoic acid is administered at a daily dose of about 400 mg. In certain embodiments, the 6,8-bis-benzylthio-octanoic acid is administered at a daily dose of about 500 mg. In certain embodiments, the 6,8-bis-benzylthio-octanoic acid is administered at a daily dose of about 600 mg. In certain embodiments, the 6,8-bis-benzylthio-octanoic acid is administered at a daily dose of about 700 mg. In certain embodiments, the 6,8-bis-benzylthio-octanoic acid is administered at a daily dose of about 800 mg. In certain embodiments, the 6,8-bis-benzylthio-octanoic acid is administered at a daily dose of about 900 mg. In certain embodiments, the 6,8-bis-benzylthio-octanoic acid is administered at a daily dose of about 1,000 mg. In certain embodiments, the 6,8-bis-benzylthio-octanoic acid is administered at a daily dose of about 1,250 mg. In certain embodiments, the 6,8-bis-benzylthio-octanoic acid is administered at a daily dose of about 1,500 mg. In certain embodiments, the 6,8-bis-benzylthio-octanoic acid is administered at a daily dose of about 1,750 mg. In certain embodiments, the 6,8-bis-benzylthio-octanoic acid is administered at a daily dose of about 2,000 mg. In certain embodiments, the 6,8-bis-benzylthio-octanoic acid is administered at a daily dose of about 2,500 mg. In certain embodiments, the 6,8-bis-benzylthio-octanoic acid is administered at a daily dose of about 3,000 mg. In certain embodiments, the 6,8-bis-benzylthio-octanoic acid is administered at a daily dose of about 3,500 mg. In certain embodiments, the 6,8-bis-benzylthio-octanoic acid is administered at a daily dose of about 4,000 mg. In certain embodiments, the 6,8-bis-benzylthio-octanoic acid is administered at a daily dose of about 4,500 mg. In certain embodiments, the 6,8-bis-benzylthio-octanoic acid is administered at a daily dose of about 5,000 mg. In certain embodiments, the 6,8-bis-benzylthio-octanoic acid is administered at a daily dose of about 6,000 mg. In certain embodiments, the 6,8-bis-benzylthio-octanoic acid is administered at a daily dose of about 7,000 mg. In certain embodiments, the 6,8-bis-benzylthio-octanoic acid is administered at a daily dose of about 8,000 mg. In certain embodiments, the 6,8-bis-benzylthio-octanoic acid is administered at a daily dose of about 9,000 mg. In certain embodiments, the 6,8-bis-benzylthio-octanoic acid is administered at a daily dose of about 10,000 mg.

The daily dose of 6,8-bis-benzylthio-octanoic acid may be administered as one dose or divided into two or more doses—e.g., b.i.d. (two times a day), t.i.d. (three times a day), or q.i.d. (four times a day). In certain embodiments, the daily dose may be split into five doses administered in regular intervals during one day. At higher daily doses and/or when administered orally or subcutaneously, it will often be beneficial to administer the daily dose of 6,8-bis-benzylthio-octanoic acid b.i.d., t.i.d., or q.i.d. Since 6,8-bis-benzylthio-octanoic acid has a relatively short half life in the blood, splitting the daily dose may improve efficacy by prolonging exposure time and may also improve safety by reducing peak plasma concentration. In certain embodiments, each dose of 6,8-bis-benzylthio-octanoic acid or pharmaceutically acceptable salt thereof is about 0.5 g to 1.5 g, and is administered once, twice, three times, four times, or five times daily. In certain embodiments, each dose of 6,8-bis-benzylthio-octanoic acid or pharmaceutically acceptable salt thereof is about 0.5 g to 1.5 g, and is administered once daily. In certain embodiments, each dose of 6,8-bis-benzylthio-octanoic acid or pharmaceutically acceptable salt thereof is about 0.5 g to 1.5 g, and is administered twice daily. In certain embodiments, each dose of 6,8-bis-benzylthio-octanoic acid or pharmaceutically acceptable salt thereof is about 0.5 g to 1.5 g, and is administered three times daily. In certain embodiments, each dose of 6,8-bis-benzylthio-octanoic acid or pharmaceutically acceptable salt thereof is about 0.5 g to 1.5 g, and is administered four times daily. In certain embodiments, each dose of 6,8-bis-benzylthio-octanoic acid or pharmaceutically acceptable salt thereof is about 0.5 g to 1.5 g, and is administered five times daily. In certain embodiments, each dose of 6,8-bis-benzylthio-octanoic acid or pharmaceutically acceptable salt thereof is about 1 g, and is administered once, twice, three times, four times, or five times daily. In certain embodiments, each dose of 6,8-bis-benzylthio-octanoic acid or pharmaceutically acceptable salt thereof is about 1 g, and is administered once daily. In certain embodiments, each dose of 6,8-bis-benzylthio-octanoic acid or pharmaceutically acceptable salt thereof is about 1 g, and is administered twice daily. In certain embodiments, each dose of 6,8-bis-benzylthio-octanoic acid or pharmaceutically acceptable salt thereof is about 1 g, and is administered three times daily. In certain embodiments, each dose of 6,8-bis-benzylthio-octanoic acid or pharmaceutically acceptable salt thereof is about 1 g, and is administered four times daily. In certain embodiments, each dose of 6,8-bis-benzylthio-octanoic acid or pharmaceutically acceptable salt thereof is about 1 g, and is administered five times daily.

The 6,8-bis-benzylthio-octanoic acid may be administered pursuant to a treatment schedule that includes days in which a dose of 6,8-bis-benzylthio-octanoic acid is administered and days in which a dose of 6,8-bis-benzylthio-octanoic acid is not administered. For example, the 6,8-bis-benzylthio-octanoic acid may be administered pursuant to a schedule in which 6,8-bis-benzylthio-octanoic acid is administered during the early days of a cycle and then not administered during the latter portion of the cycle. In certain embodiments, the 6,8-bis-benzylthio-octanoic acid is administered on days 1-5 of a 28 day cycle. In certain embodiments, the 6,8-bis-benzylthio-octanoic acid is administered on days 1, 8, and 15 of a four week cycle. In certain embodiments, the 6,8-bis-benzylthio-octanoic acid is administered on days 1 and 3 of a two week cycle. In certain embodiments, the 6,8-bis-benzylthio-octanoic acid is administered on days 1-5 of a three week cycle. In certain embodiments, the 6,8-bis-benzylthio-octanoic acid is administered on days 1-5 of a two week cycle. In certain embodiments, the 6,8-bis-benzylthio-octanoic acid is administered on days 1-3 of a three week cycle. In certain embodiments, the 6,8-bis-benzylthio-octanoic acid is administered on days 1-3 of a two week cycle. In certain embodiments, the 6,8-bis-benzylthio-octanoic acid is administered every day. In certain embodiments, the 6,8-bis-benzylthio-octanoic acid is administered every other day. In certain embodiments, the 6,8-bis-benzylthio-octanoic acid is administered three days per week. In certain embodiments, the 6,8-bis-benzylthio-octanoic acid is administered two days per week. In certain embodiments, the 6,8-bis-benzylthio-octanoic acid is administered one day per week.

In certain embodiments, hydroxychloroquine sulfate is administered at a daily dose of about 50 mg to about 1500 mg. In certain embodiments, hydroxychloroquine sulfate is administered at a daily dose of about 100 mg to about 1500 mg. In certain embodiments, hydroxychloroquine sulfate is administered at a daily dose of about 200 mg to about 1200 mg. In certain embodiments, hydroxychloroquine sulfate is administered at a daily dose of about 300 mg to about 1200 mg. In certain embodiments, hydroxychloroquine sulfate is administered at a daily dose of about 400 mg to about 1200 mg. In certain embodiments, hydroxychloroquine sulfate is administered at a daily dose of about 600 mg to about 1200 mg. In certain embodiments, hydroxychloroquine sulfate is administered at a daily dose of about 600 mg to about 1000 mg. In certain embodiments, hydroxychloroquine sulfate is administered at a daily dose of about 100 mg. In certain embodiments, hydroxychloroquine sulfate is administered at a daily dose of about 200 mg. In certain embodiments, hydroxychloroquine sulfate is administered at a daily dose of about 300 mg. In certain embodiments, hydroxychloroquine sulfate is administered at a daily dose of about 400 mg. In certain embodiments, hydroxychloroquine sulfate is administered at a daily dose of about 500 mg. In certain embodiments, hydroxychloroquine sulfate is administered at a daily dose of about 600 mg. In certain embodiments, hydroxychloroquine sulfate is administered at a daily dose of about 700 mg. In certain embodiments, hydroxychloroquine sulfate is administered at a daily dose of about 800 mg. In certain embodiments, hydroxychloroquine sulfate is administered at a daily dose of about 900 mg. In certain embodiments, hydroxychloroquine sulfate is administered at a daily dose of about 1,000 mg. In certain embodiments, hydroxychloroquine sulfate is administered at a daily dose of about 1,100 mg. In certain embodiments, hydroxychloroquine sulfate is administered at a daily dose of about 1,200 mg. In certain embodiments, hydroxychloroquine sulfate is administered at a daily dose of about 1,300 mg. In certain embodiments, hydroxychloroquine sulfate is administered at a daily dose of about 1400 mg. In certain embodiments, hydroxychloroquine sulfate is administered at a daily dose of about 1,500 mg.

In certain embodiments, hydroxychloroquine sulfate is administered at a daily dose of about 2 mg/kg to about 25 mg/kg. In certain embodiments, hydroxychloroquine sulfate is administered at a daily dose of about 5 mg/kg to about 20 mg/kg. In certain embodiments, hydroxychloroquine sulfate is administered at a daily dose of about 6.5 mg/kg to about 19.5 mg/kg. In certain embodiments, hydroxychloroquine sulfate is administered at a daily dose of about 2.5 mg/kg. In certain embodiments, hydroxychloroquine sulfate is administered at a daily dose of about 3 mg/kg. In certain embodiments, hydroxychloroquine sulfate is administered at a daily dose of about 3.5 mg/kg. In certain embodiments, hydroxychloroquine sulfate is administered at a daily dose of about 4 mg/kg. In certain embodiments, hydroxychloroquine sulfate is administered at a daily dose of about 4.5 mg/kg. In certain embodiments, hydroxychloroquine sulfate is administered at a daily dose of about 5 mg/kg. In certain embodiments, hydroxychloroquine sulfate is administered at a daily dose of about 5.5 mg/kg. In certain embodiments, hydroxychloroquine sulfate is administered at a daily dose of about 6 mg/kg. In certain embodiments, hydroxychloroquine sulfate is administered at a daily dose of about 6.5 mg/kg. In certain embodiments, hydroxychloroquine sulfate is administered at a daily dose of about 7 mg/kg. In certain embodiments, hydroxychloroquine sulfate is administered at a daily dose of about 7.5 mg/kg. In certain embodiments, hydroxychloroquine sulfate is administered at a daily dose of about 8 mg/kg. In certain embodiments, hydroxychloroquine sulfate is administered at a daily dose of about 8.5 mg/kg. In certain embodiments, hydroxychloroquine sulfate is administered at a daily dose of about 9 mg/kg. In certain embodiments, hydroxychloroquine sulfate is administered at a daily dose of about 9.5 mg/kg. In certain embodiments, hydroxychloroquine sulfate is administered at a daily dose of about 10 mg/kg. In certain embodiments, hydroxychloroquine sulfate is administered at a daily dose of about 10.5 mg/kg. In certain embodiments, hydroxychloroquine sulfate is administered at a daily dose of about 11 mg/kg. In certain embodiments, hydroxychloroquine sulfate is administered at a daily dose of about 11.5 mg/kg. In certain embodiments, hydroxychloroquine sulfate is administered at a daily dose of about 12 mg/kg. In certain embodiments, hydroxychloroquine sulfate is administered at a daily dose of about 12.5 mg/kg. In certain embodiments, hydroxychloroquine sulfate is administered at a daily dose of about 13 mg/kg. In certain embodiments, hydroxychloroquine sulfate is administered at a daily dose of about 13.5 mg/kg. In certain embodiments, hydroxychloroquine sulfate is administered at a daily dose of about 14 mg/kg. In certain embodiments, hydroxychloroquine sulfate is administered at a daily dose of about 14.5 mg/kg. In certain embodiments, hydroxychloroquine sulfate is administered at a daily dose of about 15 mg/kg. In certain embodiments, hydroxychloroquine sulfate is administered at a daily dose of about 15.5 mg/kg. In certain embodiments, hydroxychloroquine sulfate is administered at a daily dose of about 16 mg/kg. In certain embodiments, hydroxychloroquine sulfate is administered at a daily dose of about 16.5 mg/kg. In certain embodiments, hydroxychloroquine sulfate is administered at a daily dose of about 17 mg/kg. In certain embodiments, hydroxychloroquine sulfate is administered at a daily dose of about 17.5 mg/kg. In certain embodiments, hydroxychloroquine sulfate is administered at a daily dose of about 18 mg/kg. In certain embodiments, hydroxychloroquine sulfate is administered at a daily dose of about 18.5 mg/kg. In certain embodiments, hydroxychloroquine sulfate is administered at a daily dose of about 19 mg/kg. In certain embodiments, hydroxychloroquine sulfate is administered at a daily dose of about 19.5 mg/kg. In certain embodiments, hydroxychloroquine sulfate is administered at a daily dose of about 20 mg/kg.

The daily dose of hydroxychloroquine sulfate may be administered as one dose or divided into two or more doses—e.g., b.i.d. In certain embodiments, the daily dose of hydroxychloroquine sulfate is administered as one dose. In certain embodiments, the daily dose of hydroxychloroquine sulfate is divided into two doses and administered b.i.d.

In certain embodiments, chloroquine phosphate is administered at a daily dose of about 50 mg to about 2000 mg, which is equivalent to about 30 mg to about 1200 mg chloroquine base. In certain embodiments, chloroquine phosphate is administered at a daily dose of about 150 mg to about 1800 mg. In certain embodiments, chloroquine phosphate is administered at a daily dose of about 250 mg to about 1500 mg. In certain embodiments, chloroquine phosphate is administered at a daily dose of about 500 mg to about 1500 mg. In certain embodiments, chloroquine phosphate is administered at a daily dose of about 500 mg to about 1000 mg. In certain embodiments, chloroquine phosphate is administered at a daily dose of about 1000 mg to about 1500 mg. In certain embodiments, chloroquine phosphate is administered at a daily dose of about 250 mg. In certain embodiments, chloroquine phosphate is administered at a daily dose of about 500 mg. In certain embodiments, chloroquine phosphate is administered at a daily dose of about 750 mg. In certain embodiments, chloroquine phosphate is administered at a daily dose of about 1000 mg. In certain embodiments, chloroquine phosphate is administered at a daily dose of about 1,250 mg. In certain embodiments, chloroquine phosphate is administered at a daily dose of about 1,500 mg. In certain embodiments, chloroquine phosphate is administered at a daily dose of about 1750 mg. In certain embodiments, chloroquine phosphate is administered at a daily dose of about 2000 mg. In certain embodiments, chloroquine phosphate is administered at a daily dose of about 2250 mg. In certain embodiments, chloroquine phosphate is administered at a daily dose of about 2500 mg.

In certain embodiments, chloroquine phosphate is administered at a daily dose of about 2 mg/kg to about 25 mg/kg. In certain embodiments, chloroquine phosphate is administered at a daily dose of about 5 mg/kg to about 20 mg/kg. In certain embodiments, chloroquine phosphate is administered at a daily dose of about 6.5 mg/kg to about 19.5 mg/kg. In certain embodiments, chloroquine phosphate is administered at a daily dose of about 2.5 mg/kg. In certain embodiments, chloroquine phosphate is administered at a daily dose of about 3 mg/kg. In certain embodiments, chloroquine phosphate is administered at a daily dose of about 3.5 mg/kg. In certain embodiments, chloroquine phosphate is administered at a daily dose of about 4 mg/kg. In certain embodiments, chloroquine phosphate is administered at a daily dose of about 4.5 mg/kg. In certain embodiments, chloroquine phosphate is administered at a daily dose of about 5 mg/kg. In certain embodiments, chloroquine phosphate is administered at a daily dose of about 5.5 mg/kg. In certain embodiments, chloroquine phosphate is administered at a daily dose of about 6 mg/kg. In certain embodiments, chloroquine phosphate is administered at a daily dose of about 6.5 mg/kg. In certain embodiments, chloroquine phosphate is administered at a daily dose of about 7 mg/kg. In certain embodiments, chloroquine phosphate is administered at a daily dose of about 7.5 mg/kg. In certain embodiments, chloroquine phosphate is administered at a daily dose of about 8 mg/kg. In certain embodiments, chloroquine phosphate is administered at a daily dose of about 8.5 mg/kg. In certain embodiments, chloroquine phosphate is administered at a daily dose of about 9 mg/kg. In certain embodiments, chloroquine phosphate is administered at a daily dose of about 9.5 mg/kg. In certain embodiments, chloroquine phosphate is administered at a daily dose of about 10 mg/kg. In certain embodiments, chloroquine phosphate is administered at a daily dose of about 10.5 mg/kg. In certain embodiments, chloroquine phosphate is administered at a daily dose of about 11 mg/kg. In certain embodiments, chloroquine phosphate is administered at a daily dose of about 11.5 mg/kg. In certain embodiments, chloroquine phosphate is administered at a daily dose of about 12 mg/kg. In certain embodiments, chloroquine phosphate is administered at a daily dose of about 12.5 mg/kg. In certain embodiments, chloroquine phosphate is administered at a daily dose of about 13 mg/kg. In certain embodiments, chloroquine phosphate is administered at a daily dose of about 13.5 mg/kg. In certain embodiments, chloroquine phosphate is administered at a daily dose of about 14 mg/kg. In certain embodiments, chloroquine phosphate is administered at a daily dose of about 14.5 mg/kg. In certain embodiments, chloroquine phosphate is administered at a daily dose of about 15 mg/kg. In certain embodiments, chloroquine phosphate is administered at a daily dose of about 15.5 mg/kg. In certain embodiments, chloroquine phosphate is administered at a daily dose of about 16 mg/kg. In certain embodiments, chloroquine phosphate is administered at a daily dose of about 16.5 mg/kg. In certain embodiments, chloroquine phosphate is administered at a daily dose of about 17 mg/kg. In certain embodiments, chloroquine phosphate is administered at a daily dose of about 17.5 mg/kg. In certain embodiments, chloroquine phosphate is administered at a daily dose of about 18 mg/kg. In certain embodiments, chloroquine phosphate is administered at a daily dose of about 18.5 mg/kg. In certain embodiments, chloroquine phosphate is administered at a daily dose of about 19 mg/kg. In certain embodiments, chloroquine phosphate is administered at a daily dose of about 19.5 mg/kg. In certain embodiments, chloroquine phosphate is administered at a daily dose of about 20 mg/kg.

The daily dose of chloroquine phosphate may be administered as one dose or divided into two or more doses—e.g., b.i.d. In certain embodiments, the daily dose of chloroquine phosphate is administered as one dose. In certain embodiments, the daily dose of chloroquine phosphate is divided into two doses and administered b.i.d.

For simplicity, the autophagy inhibitor will typically be administered pursuant to a treatment cycle that is the same length as each treatment cycle for 6,8-bis-benzylthio-octanoic acid (e.g., 2 weeks, three weeks, four weeks, etc.). Like the cycle for 6,8-bis-benzylthio-octanoic acid, the autophagy inhibitor cycle may include days in which a dose of autophagy inhibitor is administered and days in which a dose of autophagy inhibitor is not administered. For example, the autophagy inhibitor may be administered pursuant to a schedule in which autophagy inhibitor is administered on the same days that 6,8-bis-benzylthio-octanoic acid is administered, and is not administered on the days 6,8-bis-benzylthio-octanoic acid is not administered. Alternatively, the autophagy inhibitor may be administered on some but not all days in which 6,8-bis-benzylthio-octanoic acid is administered, and/or may be administered on some but not all days on which 6,8-bis-benzylthio-octanoic acid is not administered. In certain embodiments, the autophagy inhibitor may be administered on each day of the cycle.

In certain embodiments, the dosing cycle is repeated at least once. In certain embodiments, the method of the present invention comprises treatment with two cycles or more. In certain embodiments, the method of the present invention comprises treatment with three cycles or more. In certain embodiments, the method of the present invention comprises treatment with four cycles or more. In certain embodiments, the method of the present invention comprises treatment with five cycles or more. In certain embodiments, the method of the present invention comprises treatment with six cycles or more. In certain embodiments, the method of the present invention comprises treatment with seven cycles or more. In certain embodiments, the method of the present invention comprises treatment with eight cycles or more. In certain embodiments, the method of the present invention comprises treatment with nine cycles or more. In certain embodiments, the method of the present invention comprises treatment with ten cycles or more. In certain embodiments, the method of the present invention comprises regular treatment with 6,8-bis-benzylthio-octanoic acid and an autophagy inhibitor, including on a daily or weekly basis, for an extended period of time, such as at least one month, six months, one year, two years, three years, or longer.

### Patients for Treatment

The therapeutic methods may be further characterized according to the patient to be treated. In the present invention, the patient is a human being. In certain embodiments, the patient is an adult. In certain embodiments, the patient is an adult at least 60 years of age. In certain embodiments, the patient is an adult at least 50 years of age. In certain embodiments, the patient is a child.

### Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase II or phase III clinical trial of at least 10 patients with cancer, the method of the present invention preferably provides an overall response rate of at least about 10%, a duration of response of at least about 1 month, progression-free survival (PFS) of at least about 1 month, and/or overall survival (OS) of at least about 1 month. Preferably, the phase II or phase III clinical trial comprises at least 15 patients. More preferably, the phase II or phase III clinical trial comprises at least 20 patients. More preferably, the phase II or phase III clinical trial comprises at least 25 patients. More preferably, the phase II or phase III clinical trial comprises at least 50 patients. More preferably, the phase II or phase III clinical trial comprises at least 100 patients. More preferably, the phase II or phase III clinical trial comprises at least 200 patients. More preferably, the phase II or phase III clinical trial comprises at least 300 patients. More preferably, the phase II or phase III clinical trial comprises at least 400 patients. More preferably, the phase II or phase III clinical trial comprises at least 500 patients. Preferably, the method of the present invention provides an overall response rate of at least about 20% in patients. More preferably, the method of the present invention provides an overall response rate of at least about 30%. More preferably, the method of the present invention provides an overall response rate of at least about 40%. More preferably, the method of the present invention provides an overall response rate of at least about 50%. More preferably, the method of the present invention provides an overall response rate of at least about 60%. More preferably, the method of the present invention provides an overall response rate of at least about 70%. More preferably, the method of the present invention provides an overall response rate of at least about 80%. More preferably, the method of the present invention provides an overall response rate of at least about 90%. Preferably, the method of the present invention provides a duration of response, PFS, and/or OS of at least about 2 months. Preferably, the method of the present invention provides a duration of response, PFS, and/or OS of at least about 3 months. Preferably, the method of the present invention provides a duration of response, PFS, and/or OS of at least about 4 months. Preferably, the method of the present invention provides a duration of response, PFS, and/or OS of at least about 5 months. Preferably, the method of the present invention provides a duration of response, PFS, and/or OS of at least about 6 months. Preferably, the method of the present invention provides a duration of response, PFS, and/or OS of at least about 7 months. Preferably, the method of the present invention provides a duration of response, PFS, and/or OS of at least about 8 months. Preferably, the method of the present invention provides a duration of response, PFS, and/or OS of at least about 9 months. Preferably, the method of the present invention provides a duration of response, PFS, and/or OS of at least about 10 months. Preferably, the method of the present invention provides a duration of response, PFS, and/or OS of at least about 11 months. Preferably, the method of the present invention provides a duration of response, PFS, and/or OS of at least about 12 months. Preferably, the method of the present invention provides a duration of response, PFS, and/or OS of at least about 14 months. Preferably, the method of the present invention provides a duration of response, PFS, and/or OS of at least about 16 months. Preferably, the method of the present invention provides a duration of response, PFS, and/or OS of at least about 18 months. Preferably, the method of the present invention provides a duration of response, PFS, and/or OS of at least about 20 months. Preferably, the method of the present invention provides a duration of response, PFS, and/or OS of at least about 24 months. In certain embodiments, the overall response rate, duration of response, and progression-free survival mentioned above are measured in a phase II clinical trial. In certain embodiments, the overall response rate, duration of response, and progression-free survival mentioned above are measured in a phase III clinical trial.

### EQUIVALENTS

The description above describes multiple aspects and embodiments of the invention, including therapeutic applications, treatment methods, and pharmaceutical compositions. The patent application specifically contemplates all combinations and permutations of the aspects and embodiments.

### III. EXAMPLES

The invention now being generally described, will be more readily understood by reference to the following examples, which are included merely for purposes of illustration of certain aspects and embodiments of the present invention, and are not intended to limit the invention.

### Example 1—CPI-613 Induces Autophagy in AML Cells In Vitro

K562 cells in Roswell Park Memorial Institute (RPMI) medium with 10% fetal bovine serum (FBS) (100,000 cells/mL) or MFL2 cells (Pardee, T. S. et al., Experimental Hematology, 2011, 39, 473-485) in 45% Iscove's Modified Dulbeccos' Medium (IMDM)/45% Dulbecco's Modified Eagle's Medium (DMEM)/10% FBS (50,000 cells/mL) were incubated with CPI-613 (200 μM) at 37° C. under 5% CO₂ for 4 hours alone or in the presence of early and late, respectively, autophagy inhibitors 3-methyladenine (3MA) or Bafilomycin A (BafA), and then blotted for LC3-II, which is generated during autophagy (generation reduced by 3MA) and consumed upon completion (degradation reduced by BafA). Complete media (“Full”) and Hank's Balanced Salt Solution medium (HBSS) were used as negative and positive controls of autophagy, respectively.

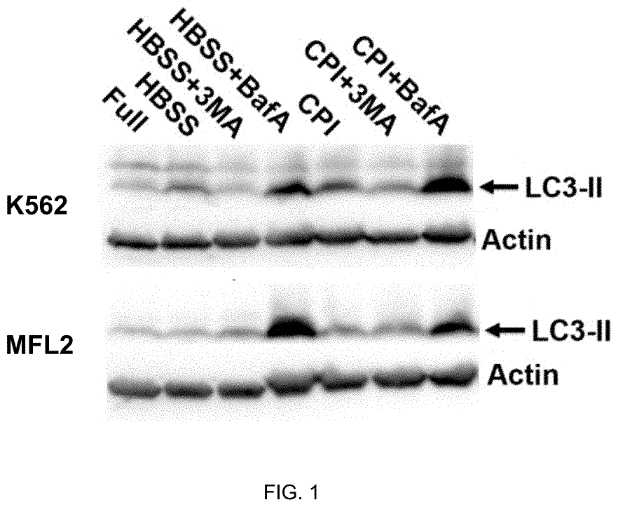

The results are presented in FIG. 1. This example demonstrates that 6,8-bis-benzylthio-octanoic acid induces autophagy in AML cells.

### Example 2—Chloroquine Sensitizes AML Cells to CPI-613 In Vitro

K562 or OCI-AML3 cells in RPMI medium with 10% FBS (100,000 cells/mL) were incubated with CPI-613 (100 μM), chloroquine (25 μM or 50 μM), or the combination of CPI-613 (100 μM) and chloroquine (25 μM or 50 μM) at 37° C. under 5% CO₂ for 72 hours and then viability assessed using the Promega CellTiter-Glo assay.

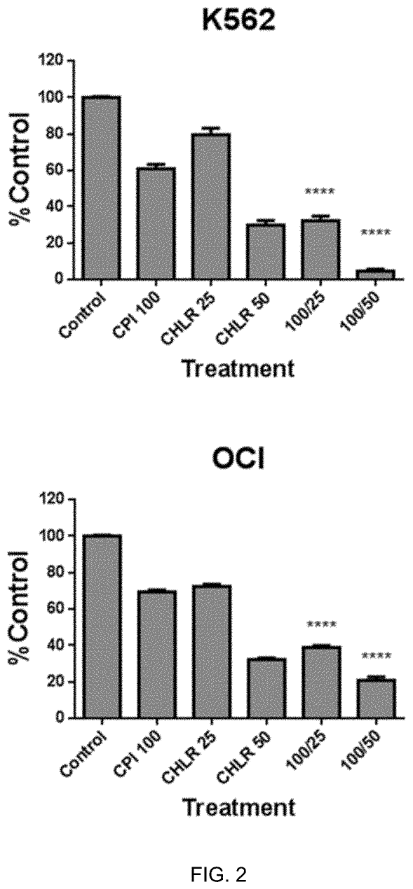

The results are presented in FIG. 2. This example demonstrates that the combination of 6,8-bis-benzylthio-octanoic acid and chloroquine kills AML cells in vitro significantly better than either agent alone.

### Example 3—Chloroquine Sensitizes AML Cells to CPI-613 In Vivo

C57Bl/6 mice were injected into their tail veins with 1 million MFL2 cells on Day 0 and beginning on Day 7, upon confirmation of engraftment by bioluminescence imaging, were treated by gavage with CPI-613 (300 mg/kg of 50 mg/mL solution in 0.05 N NaOH in 5% dextrose, adjusted to pH 7.5-8 with 4% glacial acetic acid; 1 animal) daily except weekends until death, intraperitoneally (IP) with chloroquine (200 μL (ca. 100 mg/kg) of 10 mg/mL solution in PBS; Chlr; 3 animals) daily except weekends until death, or a combination of both CPI-613 (300 mg/kg daily gavage as above) and chloroquine (200 μL daily IP as above) (4 animals) and followed for survival. The control animal (1) received both oral and IP vehicles. P value was determined by log rank test.

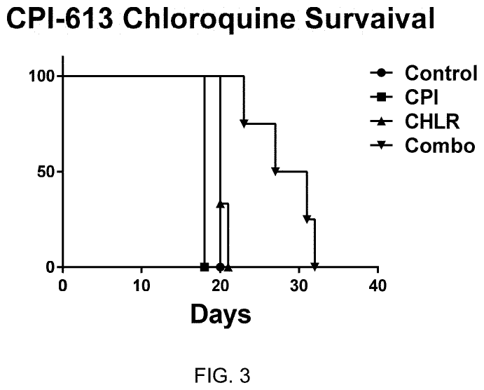

The results are presented in FIG. 3. This example demonstrates that the combination of 6,8-bis-benzylthio-octanoic acid and chloroquine significantly prolongs survival of AML tumor-bearing mice in vivo compared to either agent alone at the same concentration.

### Example 4—Metformin Sensitizes AML Cells to CPI-613 In Vitro

MFL2 cells (Pardee, T. S. et al., Experimental Hematology, 2011, 39, 473-485) in 45% IMDM/45% DMEM/10% FBS (50,000 cells/mL) were incubated with CPI-613 (50 μM), metformin (1 μM, 2.5 μM, or 5 μM), or the combination of CPI-613 (50 μM) and metformin (1 μM, 2.5 μM, or 5 μM) at 37° C. under 5% CO₂ for 72 hours and then viability assessed using the Promega CellTiter-Glo assay.

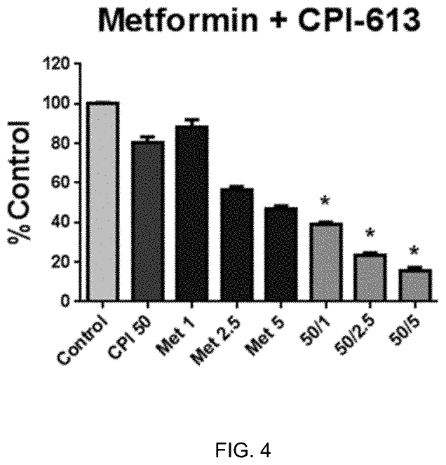

The results are presented in FIG. 4. This example demonstrates that the combination of 6,8-bis-benzylthio-octanoic acid and metformin kills AML cells in vitro significantly better than either agent alone at the same concentration.

### Example 5—Metformin Sensitizes AML Cells to CPI-613 In Vivo

C57Bl/6 mice were injected into their tail veins with 1 million MFL2 cells on Day 0 and beginning on Day 7, upon confirmation of engraftment by bioluminescence imaging, were treated with CPI-613 (300 mg/kg of a 50 mg/mL solution of CPI-613 in 0.05 N NaOH in 5% dextrose, adjusted to pH 7.5-8 with 4% glacial acetic acid) administered by daily gavage except weekends until death, metformin (1 mg/mL in the drinking water with ad lib access), or a combination of metformin (1 mg/mL ad lib in drinking water as above) plus CPI-613 (300 mg/kg daily gavage as above) (Met+CPI) and followed for survival. P value was determined by log rank test.

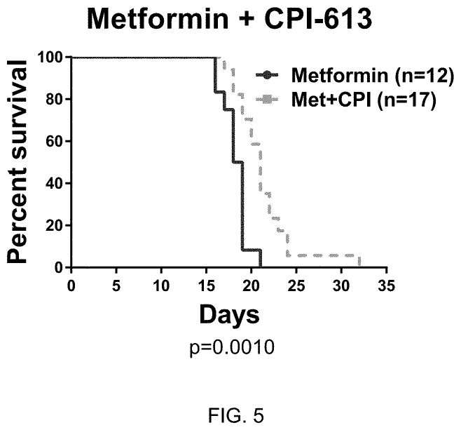

The results are presented in FIG. 5. This example demonstrates that the combination of 6,8-bis-benzylthio-octanoic acid and metformin significantly prolongs survival of AML tumor-bearing mice in vivo.

### Example 6—2-Deoxyglucose Sensitizes AML Cells to CPI-613 In Vitro

MFL2 cells (Pardee, T. S. et al., Experimental Hematology, 2011, 39, 473-485) in 45% IMDM/45% DMEM/10% FBS (50,000 cells/mL) were incubated with CPI-613 (50 μM), 2-deoxyglucose (0.25 mM or 0.5 mM), or the combination of CPI-613 (50 μM) and 2-deoxyglucose (0.25 mM or 0.5 mM) at 37° C. under 5% CO₂ for 72 hours and then viability assessed using the Promega CellTiter-Glo assay.

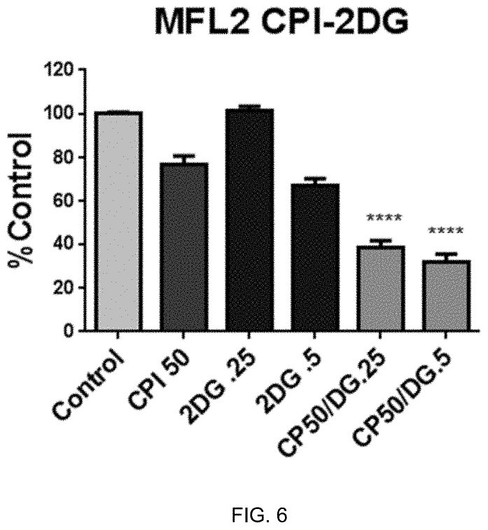

The results are presented in FIG. 6. This example demonstrates that the combination of 6,8-bis-benzylthio-octanoic acid and 2-deoxyglucose kills AML cells in vitro significantly better than either agent alone at the same concentration.

### Example 7—Combination of Chloroquine and 2-Deoxyglucose Sensitizes AML Cells to CPI-613 In Vitro

OCI-AML3 cells in RPMI medium with 10% FBS (100,000 cells/mL) or MFL2 cells (Pardee, T. S. et al., Experimental Hematology, 2011, 39, 473-485) in 45% IMDM/45% DMEM/10% FBS (50,000 cells/mL) were incubated with CPI-613 (50 μM), chloroquine (25 μM for OCI; 10 μM for MFL2), 2-deoxyglucose (10 mM for OCI; 0.25 mM for MFL2) or the combination of CPI-613 (50 μM), chloroquine (25 μM for OCI; 10 μM for MFL2), and 2-deoxyglucose (10 mM for OCI; 0.25 mM for MFL2) at 37° C. under 5% CO₂ for 72 hours and then viability assessed using the Promega CellTiter-Glo assay.

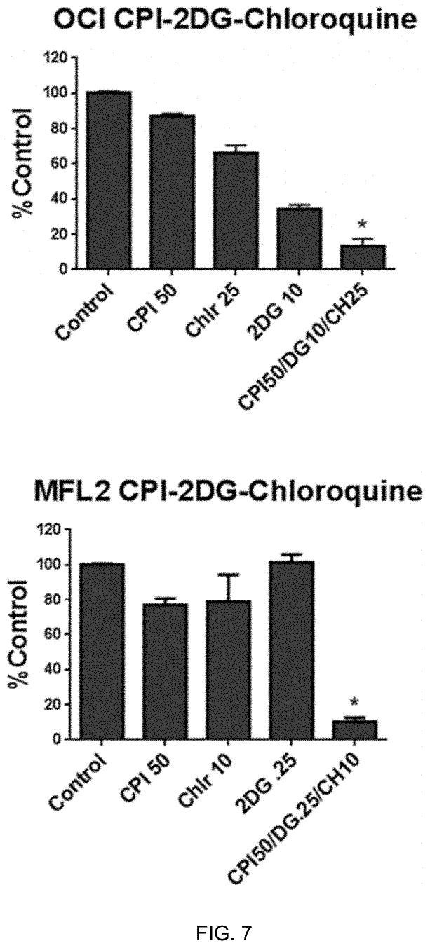

The results are presented in FIG. 7. This example demonstrates that the combination of 6,8-bis-benzylthio-octanoic acid, chloroquine, and 2-deoxyglucose kills AML cells in vitro significantly better than any of the three agents alone at the same concentration.

### Example 8—Chloroquine Sensitizes PDAC Cells to CPI-613 In Vitro

Pancreatic ductal adenocarcinoma cell (PDAC) lines, AsPC-1, PANC-1, BxPC-3, or MIA Paca-2 cells were seeded in complete RPMI medium at 30,000 cells per well in 96 well plates and incubated for 18 hrs. Cells were then adapted to nutrient depleted conditions resembling tumor nutrient conditions by incubating in RPMI without serum for 20 hrs, followed by modified Earle's Balanced Salt Solution (EBSS) (CBS2 medium) for 3 hrs. Drugs were administered in CBS2 medium and incubated for 20 hrs. Drug containing medium was replaced with RPMI without serum and incubated overnight. All incubations were in a humidified incubator at 37° C. and 5% CO₂. Cell viability was assessed with Promega CellTiter-Glo assay in which luminescence units are proportional to the number of live cells. Zero (0) luminescence units indicate that all cells were killed.

For PANC-1 and ASPC-1 cells the drug treatments were: chloroquine or hydroxychloroquine at 12.5 μM, 25 μM, 50 μM, 100 μM, or 200 μM, CPI-613 at 10 μM, 20 μM or 40 μM, or a combination of chloroquine or hydroxychloroquine with CPI-613 at these concentrations. For BxPC-3 cells the drug treatments were: chloroquine or hydroxychloroquine at 12.5 μM, 25 μM, 50 μM, 100 μM, or 200 μM, CPI-613 7.5 μM, 15 μM or 30 μM, or a combination of chloroquine or hydroxychloroquine with CPI-613 at these concentrations. For MIA PaCa-2 cells the drug treatments were: chloroquine at 12.5 μM, 25 μM, 50 μM, 100 μM, or 200 μM, CPI-613 at 10 μM, 20 μM or 40 μM, or a combination of chloroquine and CPI-613 at these concentrations.

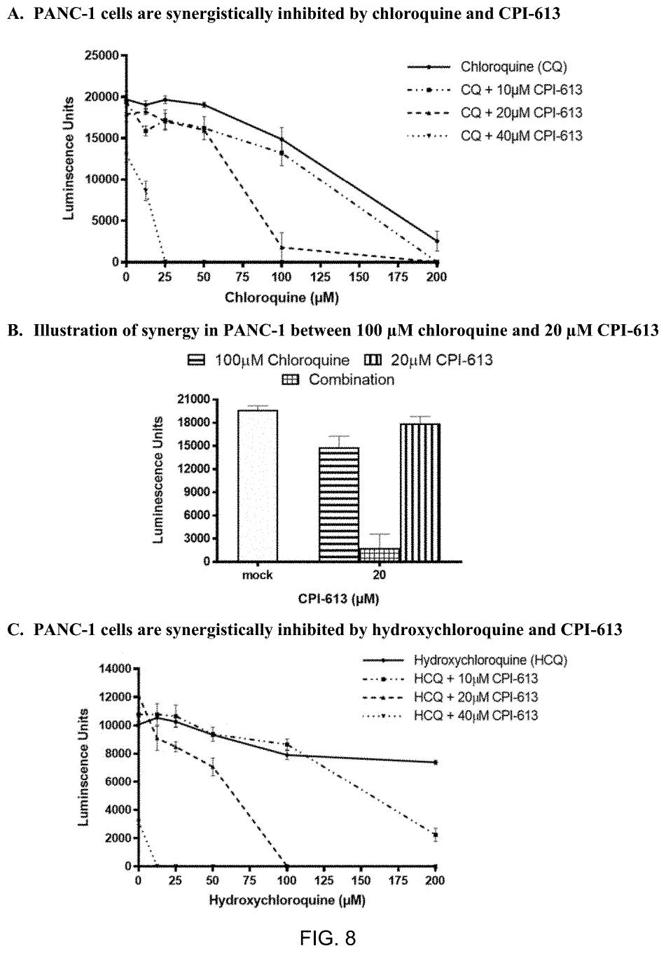

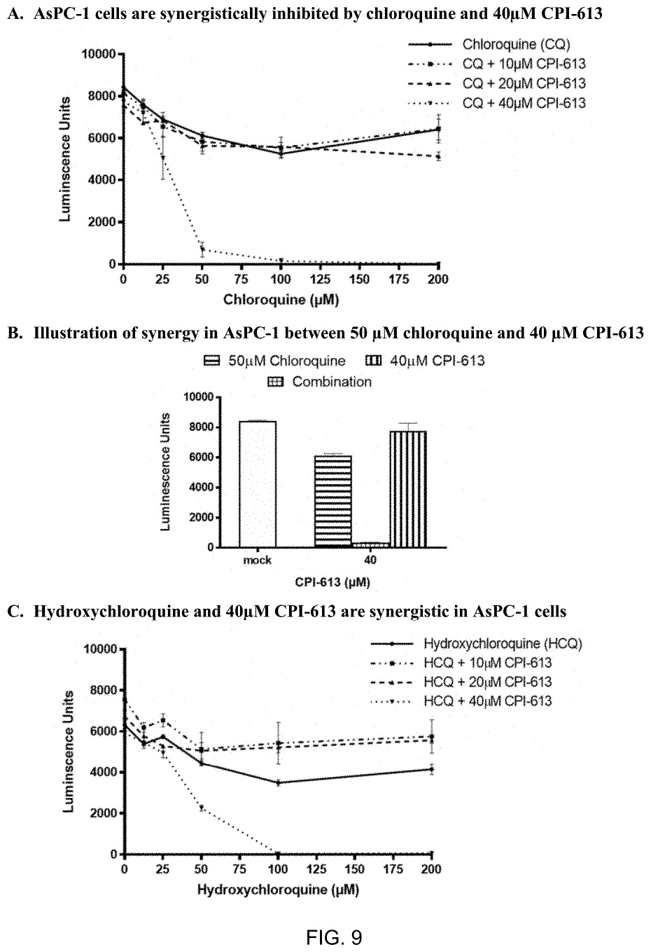

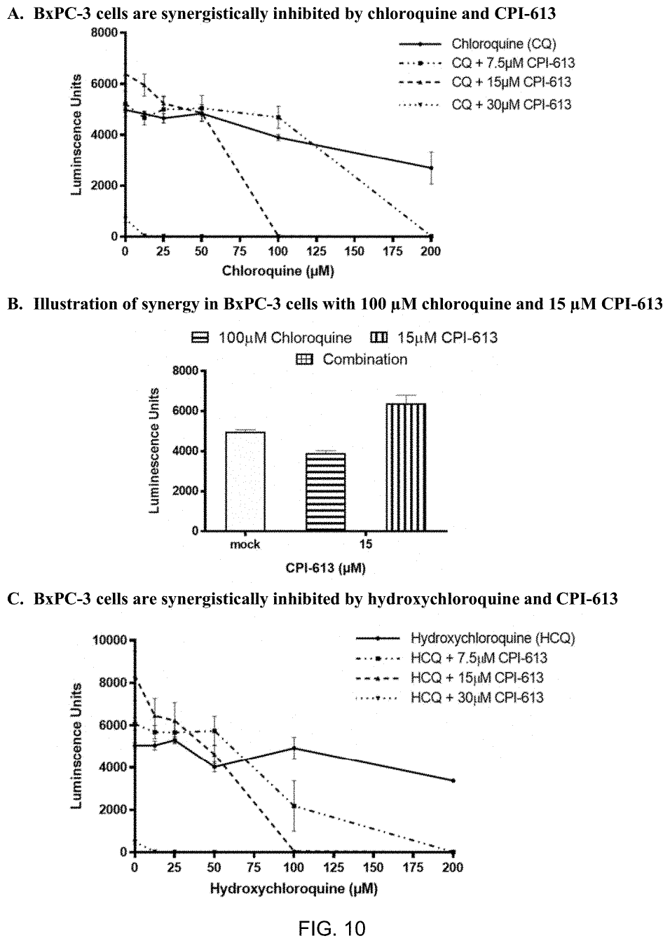

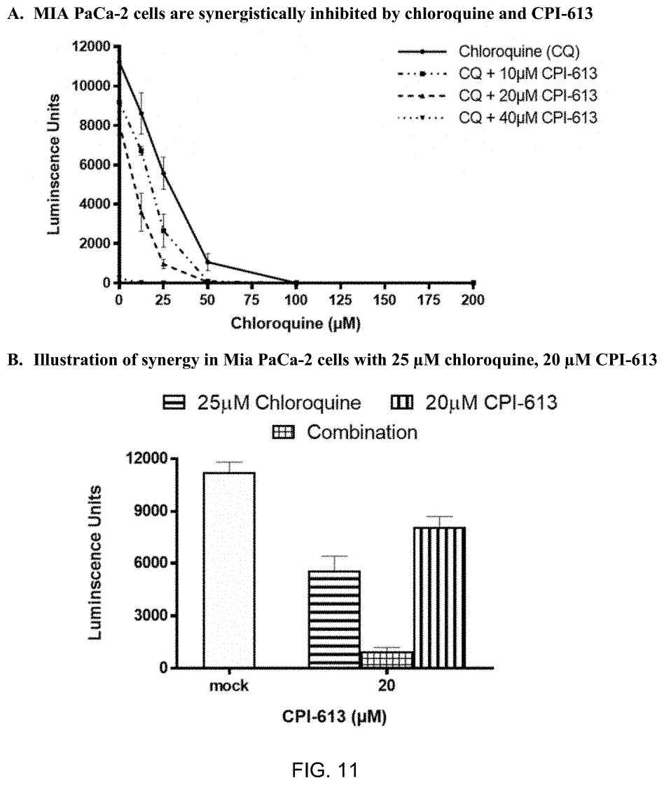

The results are presented in FIGS. 8-11. This example demonstrates that the combination of 6,8-bis-benzylthio-octanoic acid and chloroquine or hydroxychloroquine kills PDAC cells in vitro better than either agent alone.

### Example 9—Chloroquine Sensitizes Colorectal Cancer Cells to CPI-613 In Vitro

CoLo 205 cells were seeded in complete RPMI medium at 60,000 cells per well; LoVo cells were seeded in complete F12-K medium at 60,000 cells per well and SW620 and HT29 cells were seeded in complete McCoys medium at 60,000 cells per well in 96 well plates and incubated for 18 hrs. Cells were then adapted to nutrient depleted conditions which resemble tumor nutrient conditions by incubating in their respective media without serum for 20 hrs followed by modified EBSS (CBS2 medium) for 3 hrs. Drugs were administered in CBS2 medium and incubated for 20 hrs. Drug containing medium was replaced with RPMI without serum and incubated overnight. All incubations were in a humidified incubator at 37° C. and 5% CO₂. Cell viability was assessed with Promega CellTiter-Glo assay.

The drug treatments were: chloroquine at 12.5 μM, 25 μM, 50 μM or 100 μM, CPI-613 at 12.5 μM, 25 μM or 50 μM, alone or in combination.

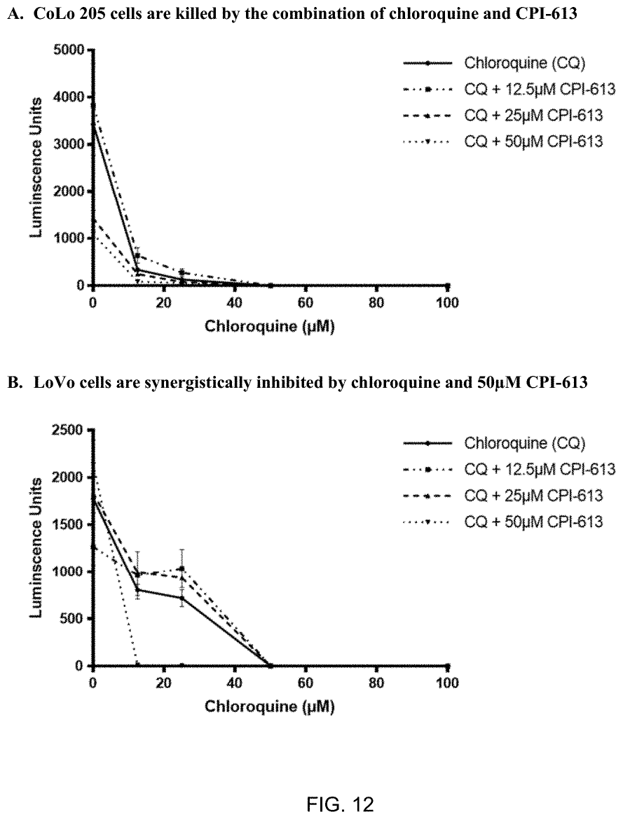

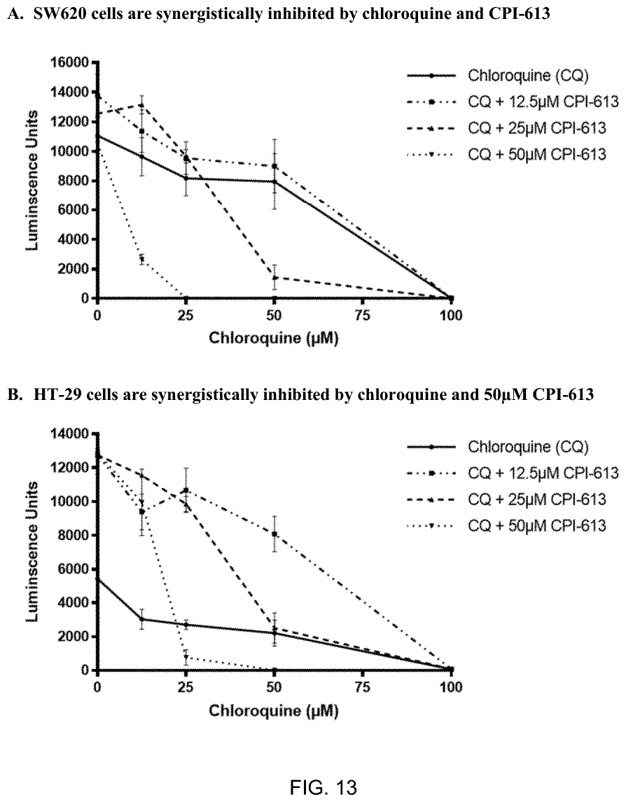

The results are presented in FIGS. 12 and 13. This example demonstrates that at the higher CPI-613 concentrations, the combination of 6,8-bis-benzylthio-octanoic acid and chloroquine kills colorectal cancer cells in vitro better than either agent alone.

### Example 10—Chloroquine Sensitizes Non-Small Cell Lung Cancer Cells to CPI-613 in Vitro

H460 cells were seeded in complete RPMI medium at 30,000 cells per well in 96 well plates and incubated for 18 hrs. Cells were then adapted to nutrient depleted conditions which resemble tumor nutrient conditions by incubating in RPMI without serum for 20 hrs followed by modified EBSS (CBS2 medium) for 3 hrs. Drugs were administered in CBS2 medium and incubated for 5 hrs. Drug containing medium was replaced with RPMI without serum and incubated overnight. All incubations were in a humidified incubator at 37° C. and 5% CO₂. Cell viability was assessed with Promega CellTiter-Glo assay.

The drug treatments were: chloroquine at 12.5 μM, 25 μM, 50 μM, 100 μM, or 200 μM, CPI-613 at 10 μM, 20 μM or 40 μM, alone or in combination.

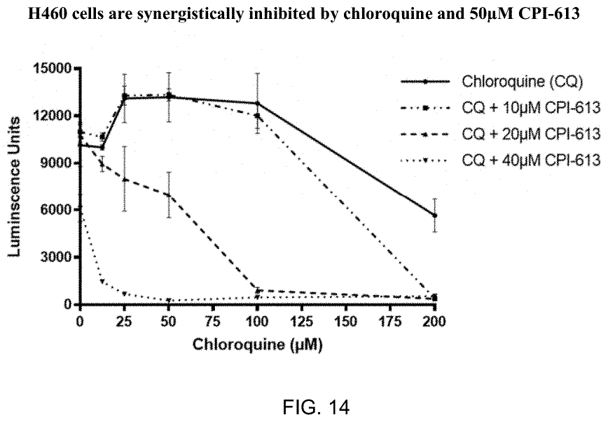

The results are presented in FIG. 14. This example demonstrates that the combination of 6,8-bis-benzylthio-octanoic acid and chloroquine kills non-small cell lung cancer cells in vitro better than either agent alone.

### Example 11—CPI-613 Induces Autophagy in HS-MM Cells In Vitro

HS-MM cells (Sonobe H. et al., J. Pathol., 1993, 169, 317-322; Sonobe H. et al., J. Pathol., 1999, 187, 594-597) were cultured with Dulbecco's modified Eagle's medium (DMEM; Gibco Life Technologies, Grand Island, N.Y., USA) containing 10% heat-inactivated fetal bovine serum (FBS). Cells were incubated with DALGreen autophagy detection agent (Dojindo Co., Kumamoto, Japan) according to the manufacturer's protocol (https://www.dojindo.com/TechnicalManual/Manual_D675.pdf). The DALGreen-treated cells were then incubated with vehicle (DMEM+10% heat-inactivated FBS containing the same amount of DMSO and distilled water present in the corresponding CPI-613 and chloroquine experiments), CPI-613 (added 0.24 of a 5 mg/mL solution of CPI-613 in DMSO per mL of medium to provide a 1 μg/mL concentration of CPI-613 in the medium), or the combination of CPI-613 (1 μg/mL as above) and chloroquine (added 2 μL of a 5 mg/mL solution of chloroquine in distilled water per mL of medium to provide a 10 μg/mL concentration of chloroquine in the medium) at 37° C. under 5% CO₂ for 16 hours, and then visualized under a confocal fluorescence microscope (Leica TCS SP8; Leica Corporation, Germany) and analyzed with a Guava Easy-Cyte cell analyzer (Haward, Calif., USA).

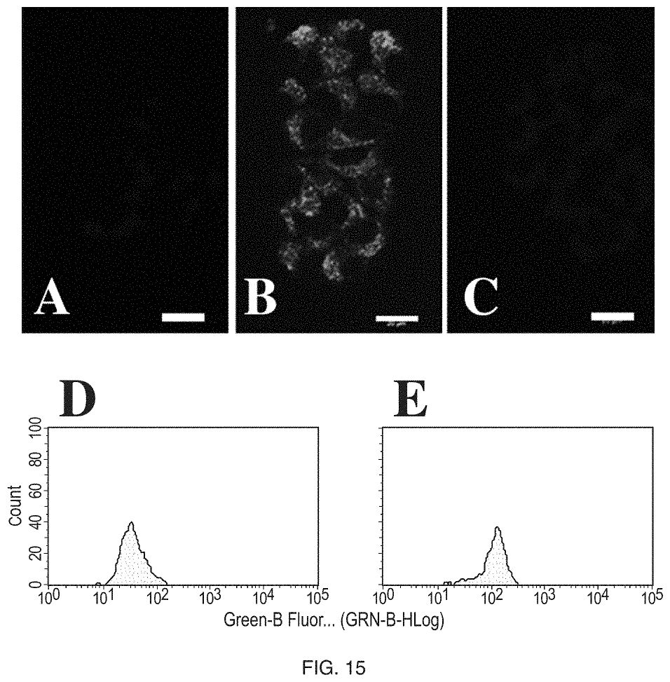

The results are presented in FIG. 15. This example demonstrates that 6,8-bis-benzylthio-octanoic acid induces autophagy in HS-MM cells, and that the autophagy induction can be blocked with chloroquine.

### Example 12—Chloroquine Sensitizes HS-MM Cells to CPI-613 In Vitro

HS-MM cells (Sonobe H. et al., J. Pathol., 1993, 169, 317-322; Sonobe H. et al., J. Pathol., 1999, 187, 594-597) were cultured with Dulbecco's modified Eagle's medium (DMEM; Gibco Life Technologies, Grand Island, N.Y., USA) containing 10% heat-inactivated fetal bovine serum (FBS). Cells were incubated with vehicle (DMEM+10% heat-inactivated FBS containing the same amount of DMSO and distilled water present in the corresponding CPI-613, chloroquine, and necrostatin-1 experiments), chloroquine (10 μg/mL), CPI-613 (1 μg/mL or 10 μg/mL), the combination of chloroquine (10 μg/mL) and CPI-613 (100 ng/mL, 1 μg/mL, or 10 μg/mL), or the combination of chloroquine (10 μg/mL) and CPI-613 (1 μg/mL) in the presence of necrostatin-1 (5 μM, 10 μM, or 50 μM), and then double stained with a fluorescein isothiocyante (FITC)-conjugated Annexin V and propidium iodide (PI) (PromoCell GMbH, Heidelberg, Germany) followed by analysis with a confocal fluorescence microscope and cell analyzer. The chloroquine and CPI-613 were added to the media from 5 mg/mL stock solutions in distilled water or DMSO, respectively, as in Example 11. The necrostatin-1 was added to the media from a 5 mg/mL stock solution in DMSO.

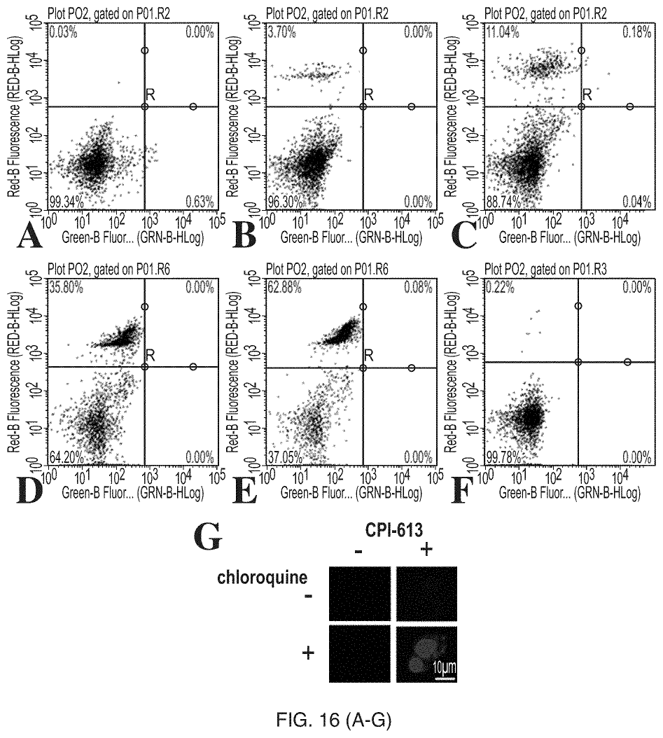

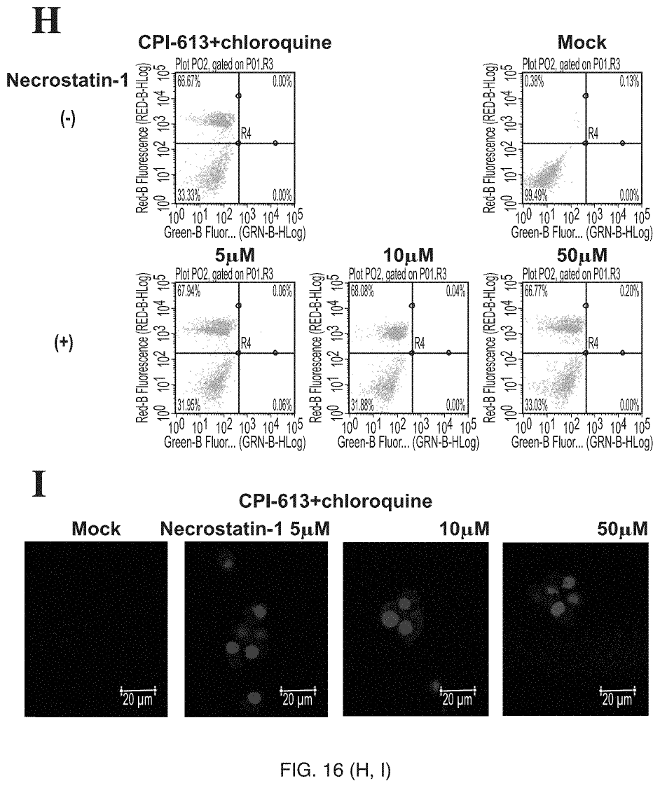

The results are presented in FIG. 16. This example demonstrates that 6,8-bis-benzylthio-octanoic acid and chloroquine kills HS-MM cells in vitro significantly better than either agent alone. A necroptosis inhibitor, necrostatin-1, did not affect cell death, noted as Annexin V negative and PI positive, indicating that CPI-613 and chloroquine induced necrotic cell death.

### Example 13—Chloroquine Sensitizes HS-MM Cells to CPI-613 In Vivo

SCID-beige (CB17.Cg-PrkdcscidLystbg-J/CrlCrlj) mice purchased from Charles River Laboratories Japan (Sizuoka, Japan) were injected into the aponeuroses of the thighs with 25 million HS-MM cells in 0.5 mL PBS on Day 0. About two weeks later, when tumor volumes reached approximately 2 mm³ (measured by calipers using the following equation: tumor volumes (mm³)=4/3π×[a/2]×[b/2]², where ‘a’ and ‘b’ correspond to the longest and shortest diameter) the mice were intraperitoneally (IP) administered twice per week for two weeks vehicle (same vehicle as used for the CPI-613 injections), CPI-613 (25 mg/kg), chloroquine (50 mg/kg), or a combination of both CPI-613 (25 mg/kg as above) and chloroquine (50 mg/kg as above). The chloroquine monotherapy solution was prepared by adding the required amount (based on the animal's weight) of chloroquine from a 5 mg/mL stock solution in distilled water to 0.5 mL of PBS to provide an intermediate solution, and then adding another 0.5 mL PBS to the intermediate solution prior to injection. The CPI-613 monotherapy solution was prepared by gradually adding the required amount (based on the animal's weight) of CPI-613 from a 10 mg/mL stock solution in DMSO to warmed 0.5 mL PBS with continuous vortexing to provide an intermediate solution, and then adding another 0.5 mL PBS to the intermediate solution prior to injection. The chloroquine/CPI-613 combination therapy solution was prepared by combining the 0.5 mL chloroquine and 0.5 mL CPI-613 intermediate solutions prior to injection. One week after the last injection, the mice were sacrificed to examine their metastatic status. The experiment was repeated under similar conditions using male SCID-beige mice.

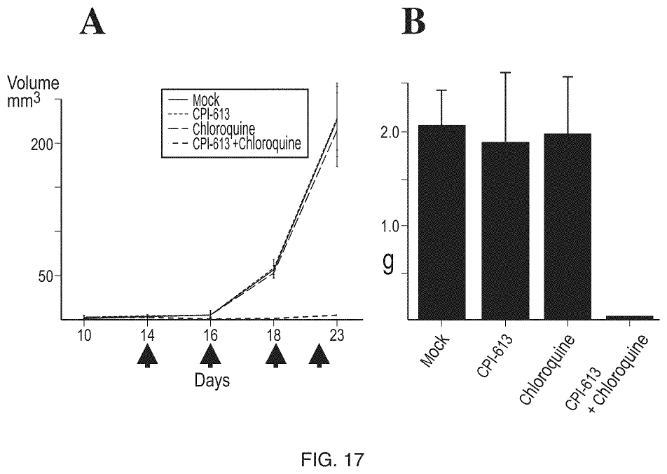

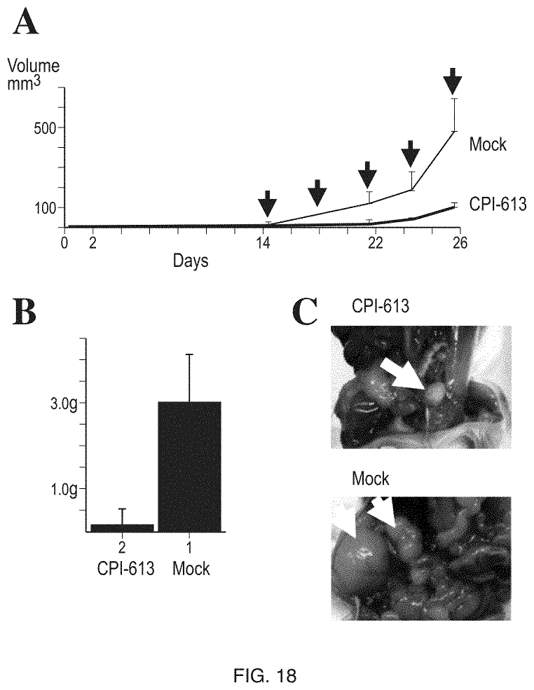

The results are presented in FIGS. 17 and 18. This example demonstrates that the combination of 6,8-bis-benzylthio-octanoic acid and chloroquine significantly decreased tumor growth in an orthotropic metastatic tumor model of CCS at the injection site compared to control (Student's t-test, P<0.01 at days 18 and 23), and significantly reduced intraperitoneal metastasis compared to control (Student's t-test, P<0.01).

### Example 14—Treatment of High Risk MDS in Human Patients Who have Failed Hypomethylating Therapy Using a Combination of CPI-613 and Hydroxychloroquine

This is a single-arm open-label study. The investigators and study subjects are not blinded to the treatment. Also, the assignment of patients will not be randomized, since there is only a single arm in this study.

The primary objective of this study is to determine the overall response rate (complete remission (CR), marrow CR, partial remission (PR), Hematologic improvement (HI)) of high risk MDS patients who have failed hypomethylating agents, treated with the combination of CPI-613 and the maximally tolerated dose of hydroxychloroquine (HCQ). Secondary objectives are to evaluate the safety of the combination, progression-free-survival (PFS), overall survival (OS) defined as the time from enrolment on study to death from any cause, and changes in the frequency of blood transfusions.

### Study Design

The dose of CPI-613 will be 2,000 mg/m². The maximal tested dose of HCQ will be 1,200 mg. The sample size will be a total of 17 patients treated at the MTD of HCQ for this Phase ½ trial. This number is based on Simon's two stage design where 9 patients will be enrolled in stage 1. If none of the 9 patients have a response the study will be stopped for lack of efficacy. If one or more patients has a response the trial will continue until a total of 17 patients have been treated with the combination at the MTD of HCQ. If 2 or more of the 17 patients have a response the combination will be considered of sufficient activity to merit additional study.

The initial phase of the study will be a dose escalation of hydroxychloroquine from 600 mg to 1200 mg PO flat dose given 2 hours before the CPI-613 infusion on days 1-5 of every 28. The dose of the CPI-613 will be 2,000 mg/m² and will not be escalated. Cohorts of 3 patients each will be treated with 600, then 800 then 1,200 mg of HCQ in a 3+3 dose escalation design as described below.

If no patients in a given cohort develop a dose-limiting toxicity (See DLT definition below), dose escalation will continue in cohorts of 3 patients. However, if a DLT is observed in a patient (whether it is the first, second or third of the 3 intended patients) at any dose level, the cohort of that dose level will be expanded to a maximum of 6 patients. If no DLT is observed in another patient out of a maximum of 6 patients, dose escalation procedure will continue in 3 patients for each subsequent cohort until a final dose of 1,200 mg is reached. However, once a DLT is observed in a total of 2 patients at any dose level, dosing of HCQ in patients at that dose level will stop immediately, even though the total number of patients at the last cohort may be as few as 2. Dose escalation is considered to be complete. The dose level that induces a DLT in 2 or more patients is considered to be above MTD, and the dose level immediately below the dose level that induced a DLT in ≥2 patients is considered the MTD. If the initial dose of 600 mg proves above the MTD the HCQ dose will drop to dose level −1 (400 mg). Should that dose (400 mg) produce 2 DLTs the study will close and the toxicity data reviewed prior to any additional enrollment. Should the 1,200 mg cohort be completed without a DLT in the first 3 patients or without a second DLT in the first 6 patients, dose escalation of the HCQ will be complete. All subsequent patients will be treated at this dose level. HCQ dose levels are summarized in Table 1.

TABLE 1

| Dose level | HCQ Dose (flat) |
|---|---|
| −1 | 400 mg |
| 1 | 600 mg |
| 2 | 800 mg |
| 3 | 1,200 mg |

Definition of DLT: A dose limiting toxicity is defined as the occurrence of any clinically relevant, grade ≥3 toxicity attributed as probably or definitely related to the combination of HCQ and CPI-613. The following toxicities are excluded from defining a DLT: grade 3 nausea and vomiting responsive to anti-emetics, grade 3 diarrhea responsive to anti-diarrheal therapy, grade 3 tumor lysis syndrome, grade 3 or 4 metabolic derangements attributed to tumor lysis syndrome or antimicrobial medications that correct with oral or IV supplementation. Hematologic toxicities can only serve as a DLT if they represent a significant (≥50%) decline from baseline values. Infectious toxicities can only serve as a DLT if they are a consequence of a ≥20% decline in ANC from baseline in the opinion of the treating investigator and not a consequence of the natural history of relapsed or refractory MDS.

### Inclusion Criteria

Patients must meet all of the following inclusion criteria before enrollment:
1) Histologically documented MDS whose disease has failed to respond, progressed or relapsed while on a hypomethylating agent.
1) Revised International Prognostic Scoring System (IPSS-R) score of Intermediate, high or very high at time of enrollment
2) ECOG Performance Status of ≤3.
3) Men and women 18 years of age or older.
4) Expected survival >2 months.
5) Women of child-bearing potential (i.e., women who are pre-menopausal or not surgically sterile) must use accepted contraceptive methods (abstinence, intrauterine device [IUD], oral contraceptive or double barrier device) during the study, and must have a negative serum or urine pregnancy test within 1 week prior to treatment initiation.
6) Fertile men must practice effective contraceptive methods during the study, unless documentation of infertility exists.
7) Patients must have fully recovered from the acute, non-hematological, non-infectious toxicities of any prior treatment with cytotoxic drugs, radiotherapy or other anti-cancer modalities. Patients with persisting, non-hematologic, non-infectious toxicities from prior treatment ≤Grade 2 are eligible, but must be documented as such.
8) Laboratory values obtained ≤2 weeks prior to enrollment must demonstrate adequate hepatic function, renal function, and coagulation as defined below:
   a. aspartate aminotransferase [AST/SGOT]≤3× upper normal limit [UNL]
   b. alanine aminotransferase [ALT/SGPT]<3×UNL
   c. bilirubin ≤2×UNL
   d. serum creatinine ≤1.5 mg/dL or 133 μmol/L
   e. albumin >2.0 g/dL or >20 g/L.
9) Mentally competent, ability to understand and willingness to sign an IRB-approved written informed consent form.
10) Have access via central line (e.g., portacath).

### Exclusion Criteria

Patients with the following characteristics are excluded:
1) Serious medical illness, such as significant cardiac disease (e.g. symptomatic congestive heart failure, unstable angina pectoris, symptomatic coronary artery disease, myocardial infarction within the past 3 months, uncontrolled cardiac arrhythmia, pericardial disease or New York Heart Association Class III or IV), or severe debilitating pulmonary disease, that would potentially increase patients' risk for toxicity.
2) Patients with active central nervous system (CNS) or epidural tumor.
3) Any active uncontrolled bleeding or bleeding diathesis (e.g., active peptic ulcer disease).
4) Any condition or abnormality which may, in the opinion of the investigator, compromise his or her safety.
5) Pregnant women, or women of child-bearing potential not using reliable means of contraception.
6) Fertile men unwilling to practice contraceptive methods during the study period.
7) Lactating females.
8) Life expectancy less than 2 months.
9) Unwilling or unable to follow protocol requirements.
10) Evidence of ongoing uncontrolled serious infection.
11) Requirement for immediate palliative treatment of any kind including surgery.
12) Patients with uncontrolled HIV infection.
13) Patients who have received radiotherapy, surgery, treatment with cytotoxic agents (except a hypomethylating agent, i.e. azacytidine or decitabine), treatment with biologic agents, immunotherapy, or any other anti-cancer therapy of any kind, or any other standard or investigational treatment for their cancer, or any other investigational agent for any indication, within the past 2 weeks prior to initiation of CPI-613 treatment.
14) Patients that have received a chemotherapy regimen with stem cell support in the previous 6 months

### Study Procedures

Table 2 provides an overview of assessments and procedures conducted during the pre-study screen and during each treatment cycle.

TABLE 2

| | | Each Treatment Cycle = 4 weeks | | | | | | |
|---|---|---|---|---|---|---|---|---|
| | Pre- | | | | | | Last day | Follow- |
| Assessments | study⁸ | D1 | D2 | D3 | D4 | D5 | of cycle | Up |
| Medical history | √ | | | | | | | |
| Physical exam, | √ | | | | | | | |
| height, weight, vitals | | | | | | | | |
| Pregnancy test | √ | | | | | | | |
| Evaluation of | √ | √ | √ | √ | √ | √ | | |
| symptoms and vital | | | | | | | | |
| signs | | | | | | | | |
| ECOG performance | √ | | | | | | | |
| status and survival | | | | | | | | |
| Clinical chemistry, | √ | √² | √² | √² | √² | √² | | |
| hematology^{3,6} | | | | | | | | |
| CPI-613¹ | | √ | √ | √ | √ | √ | | |
| Hydroxychloroquine⁹ | | √ | √ | √ | √ | √ | | |
| Assessment of | | | | | | | √⁴ | |
| response^{6,7} | | | | | | | | |
| Bone marrow exam⁶ | √ | | | | | | √⁴ | |
| Optional Blood and | √ | √ | √ | √ | √ | √ | | |
| Marrow Aspirate | | | | | | | | |
| Samples¹⁰ | | | | | | | | |
| Survival and post- | | | | | | | | √ |
| study follow-up⁵ | | | | | | | | |

D = day,
ECOG = Eastern Cooperative Oncology Group;
hr = hour;
min = minute.
¹CPI-613 is given as a 2-hr IV infusion via a central venous catheter.
²Creatinine must be performed with results available for review before administration of the CPI-613.
³Specific chemistry and hematology are listed in section 5.2.2. Renal function will be assessed utilizing the Cockcroft-Gault formula.
4 Cycles 3, 6 and 12 (and every 6 months thereafter and at disease progression when possible).
5 Survival and post-study cancer treatment will be monitored by the treating physician during routine follow-up visits for 5 years or until death.
6 CR, marrow CR, and PR will be assessed using hematology results from blood work and bone marrow exam results, according to the criteria described Cheson et al. (2006).
⁷Frequency of transfusion is defined as the number of transfusions received during the previous 8 weeks. The baseline assessment should reflect the number of transfusions received the 8 weeks prior to enrollment. On-treatment assessments should be performed at the end of cycles 4, 8 and 12 (and every 6 months thereafter until disease progression)
⁸Pre-study requirements must be performed within the following time frames: Within 4 weeks: bone marrow exam; Within 2 weeks: medical history, physical exam, vital signs, height, weight, ECOG, evaluation of symptoms and medications, clinical chemistry, hematology, and coagulation; Within 1 week: pregnancy test for women of child-bearing potential and frequency of transfusion.
⁹Hydroxychloroquine to be taken 2 hour prior to CPI-613 infusion.
¹⁰3-5 ml whole blood sample in an EDTA tube taken prior to and at completion of the CPI-613 infusion during cycle 1 only. 3-5 ml of marrow aspirate in a heparinized tube (green top) taken at the time of the bone marrow exam (see footnote 4). Samples are optional and patients who have had a baseline marrow prior to consenting for study do not need it repeated to enroll.

Treatment with CPI-613 and Hydroxychloroquine

CPI-613 will be administered to patients as shown in Table 3. Briefly, CPI-613 is given on Days 1 through 5 of a 28 day cycle with hydroxychloroquine taken orally 2 hours prior to each dose. Patients will receive pre-treatment antiemetics and supportive measures as determined by their treating physician. The default premedications will consist of ondansetron 16 mg either IV infused 15 minutes or oral 30 minutes prior to therapy and loperamide 2 mg orally (unless patient has not had a bowel movement in the last 48 hours) 30 min prior to therapy.

Baseline transfusion frequency should be recorded as the number of transfusions received during the 8 weeks prior to enrollment. Once patients have started protocol treatment, transfusion frequency should be assessed at the end of every 2 treatment cycles.

TABLE 3

| Treatment Cycles | | | Administration | Administration of |
|---|---|---|---|---|
| | Week | Day | of HCQ | CPI-613 |
| Cycle 1 and | 1 | 1-5 | Taken orally 2 | 2-hr IV infusion |
| beyond (4 | | | hours prior to | via a central |
| weeks) | | | CPI-613 infusion | venous catheter |
| | | 6-7 | Rest | Rest |
| | 2-4 | 8-28 | Rest | Rest |
| | 4 | | | Bone marrow biopsy |
| | | | | (week 4 of cycles |
| | | | | 3, 6 and 12 then |
| | | | | every 6 cycles |
| | | | | and at disease |
| | | | | progression) |

Safety Assessment

The safety of CPI-613 will be assessed from the first dose to 1 month after last dose of CPI-613. The assessment will be based on: evaluation of symptoms, vital signs, ECOG performance status and survival, clinical chemistry (and renal function utilizing the Cockcroft-Gault formula), and hematology. All safety assessment tests are performed during screening (performed within 2 weeks prior to treatment with CPI-613). Additionally, evaluation of symptoms, vital signs, ECOG and survival will be assessed on each treatment day, with results available for review within 24 hours before administration CPI-613. Clinical chemistry (renal function utilizing the Cockcroft-Gault formula) hematology, and coagulation will be performed on Day 1 of each treatment cycle, with results available for review within 24 hours before administration CPI-613.

For toxicities attributed as at least possibly related to CPI-613 dose adjustments will be as outlined in the following Table 4.

TABLE 4

| | |
|---|---|
| Toxicity and Intensity | Supportive Care and Dose Adjustment Guidelines |

Nausea, Acute (common)

| | |
|---|---|
| Grade 1 or 2 | Maintain dose and schedule. Rule out other causes. Utilize anti- |
| (If intolerable or persistent | nausea medications including 5-HT3 antagonists. |
| Grade 2 not responsive to | |
| supportive care, follow | |
| guidelines for Grade 3) | |
| Grade 3-4 | Rule out other causes. Utilize anti-nausea medications including |
| | 5-HT3 antagonists. |
| | Interrupt CPI-613 dosing until resolved to Grade 1 or baseline |
| | and reduce CPI-613 dose by 50%. |

Diarrhea (common)

| | |
|---|---|
| Grade 1 | Maintain dose and schedule. Rule out other causes including |
| | drug effects. Treat per institutional guidelines with anti-diarrheals. |
| Grade 2 | Rule out other causes including drug effects. Treat per |
| | institutional guidelines with anti-diarrheals. Interrupt CPI-613 |
| | dosing until resolved to Grade 1 or baseline. |
| | For first occurrence, restart CPI-613 at current dose. |
| | For ≥ second occurrence, reduce dose by 25%. |
| Grade 3 or 4 | Interrupt CPI-613 dosing until resolved to Grade 1 or baseline |
| | and patient is clinically stable. Reduce CPI-613 dose by 50%. |

Renal Failure

| | |
|---|---|
| Grade 1 | Maintain dose and schedule. |
| Grade 2 | Hold dose until resolved to grade 1 or baseline and resume CPI- |
| | 613 at a 25% dose reduction. |
| Grade ≥ 3 | Hold dose until resolved to grade 1 or baseline and resume CPI- |
| | 613 at a 50% dose reduction. |

Non-Hematological Adverse Events

| | |
|---|---|
| Grade 1 | Maintain dose and schedule. |
| Grade 2 | Hold dose until resolved to grade 1 or baseline and resume CPI- |
| | 613 at a 25% dose reduction. |
| Grade 3 or 4 | Hold dose until resolved to grade 1 or baseline and resume CPI- |
| | 613 at a 50% dose reduction. |

Hematological Adverse Events

| | |
|---|---|
| Grade 1-3 | Maintain dose and schedule. Utilize growth factor and transfusion |
| | supportive care asper institutional guidelines. |
| Grade 4 lasting < 7 days | Maintain dose and schedule. Utilize growth factor and transfusion |
| | supportive care asper institutional guidelines. |
| Grade 4 lasting ≥ 7 days | Hold dose until resolved to grade 3 or less and resume CPI-613 |
| | at a 50% dose reduction. |

Once toxicities have resolved to less than grade 1 (or returned to baseline) patients can have the dose of CPI-613 re-escalated at the discretion of the treating investigator. Patients that have recurrence of the original toxicity are not then eligible for dose re-escalation.

Assessment of Response

Tumor response will be assessed based on RR, PFS, and OS (as described by Cheson B. D. et al., Clinical application and proposal for modification of the International Working Group (IWG) response criteria in myelodysplasia. Blood. 108:419-425, 2006), as well as changes in the frequency of transfusion from baseline. RR and PFS, derived from hematology and bone marrow exam, will be assessed at baseline, during week 4 of cycles 3, 6, and 12 and then every 6 treatment cycles thereafter until disease progression.

Baseline transfusion frequency should be recorded as the number of transfusions received during the 8 weeks prior to enrollment. Once patients have started protocol treatment, transfusion frequency should be assessed at the end of every 2 treatment cycles.

Survival will be assessed during the study and will be monitored by treating physician contact after the patients are taken off the trial. The ECOG Performance Status scales (Oken M. M. et al., Toxicity And Response Criteria Of The Eastern Cooperative Oncology Group. Am J Clin Oncol 5:649-655, 1982) will be used to assess how a patient's disease is progressing and assess how the disease affects the daily living abilities of the patient. ECOG Grade 0=Normal activity. Fully active, able to carry on all pre-disease performance without restriction. ECOG Grade 1=Symptoms, but ambulatory; restricted in physically strenuous activity, but ambulatory and able to carry out work of a light or sedentary nature (e.g., light housework, office work). ECOG Grade 2=in bed <50% of the time; ambulatory and capable of all self-care, but unable to carry out any work activities; up and about more than 50% of waking hours. ECOG Grade 3=In bed >50% of the time; capable of only limited self-care, confined to bed or chair more than 50% of waking hours. ECOG Grade 4=100% bedridden; completely disabled; cannot carry on any self-care; totally confined to bed or chair. ECOG Grade 5=dead.

The following parameters from bone marrow exam will be recorded: morphology, immunophenotype, cellularity, karyotype (cytogenetics and FISH as applicable), molecular markers, % of bone marrow myeloblasts, % of dysplasia, WHO classification.

The Modified International Working Group (IWG)-2006 response criteria for altering natural history of MDS are described in the following Table 5.

TABLE 5

| Category | Response criteria (responses must last at least 4 wk) |
|---|---|
| Complete | Bone marrow: ≤ 5% myeloblasts with normal maturation of all cell lines* |
| remission | Persistent dysplasia will be noted*^{†} |
| | Peripheral blood^{‡} |
| | Hgb ≥ 11 g/dL |
| | Platelets ≥ 100 × 10⁹/L |
| | Neutrophils ≥ 1.0 × 10⁹/L^{†} |
| | Blasts 0% |
| Partial | All CR criteria if abnormal before treatment except: |
| Remission | Bone marrow blasts decreased by ≥ 50% over pretreatment but still > 5% |
| | Cellularity and morphology not relevant |
| Marrow CR^{†} | Bone marrow: ≤ 5% myeloblasts and decrease by ≥ 50% over pretreatment^{†} |
| | Peripheral blood: if HI responses, they will be noted in addition to marrow CR^{†} |
| Stable disease | Failure to achieve at least PR, but no evidence of progression for > 8 wks |
| Failure | Death during treatment or disease progression characteized by worsening of |
| | cytopenias, increase in percentage of bone marrow blasts, or progression to a |
| | more advanced MDS FAB subtype than pretreatment |
| Relapse after | At least 1 of the following: |
| CR or PR | Return to pretreatment bone marrow blast percentage |
| | Decrement of ≥ 50% from maximum remission/response levels |
| | in granulocytes or platelets |
| | Reduction in Hgb concentration by ≥ 1.5 g/dL or transfusion dependence |
| Cytogenetic | Complete |
| response | Disappearance of the chromosomal abnormality without appearance of new |
| | ones |
| | Partial |
| | At least 50% reduction of the chromosomal abnormality |
| Disease | For patients with: |
| progression | Less than 5% blasts: ≥ 50% increase in blasts to > 5% blasts |
| | 5%-10% blasts: ≥ 50% increase to > 10% blasts |
| | 10%-20% blasts: ≥ 50% increase to > 20% blasts |
| | 20%-30% blasts: ≥ 50% increase to > 30% blasts |
| | Any of the following: |
| | At least 50% decrement from maximum remission/response in granulocytes or |
| | platelets |
| | Reduction in Hgb by ≥ 2 g/dL |
| | Transfusion dependency |
| Survival | Endpoints: |
| | Overall: death from any cause |
| | Event free: failure or death from any cause |
| | PFS: disease progression or death from MDS |
| | DFS: time to relapse |
| | Cause-specific death: death related to MDS |

To convert hemoglobin from grams per deciliter to mins per liter, multiply grams per deciliter by 10.
MDS indicates myelodysplastie syndromes;
Hgb, hemoglobin;
CR, complete remission;
HI, hematologic improvement;
PR, partial remission;
FAB, French-American-British;
AML, acute myeloid leukemia;
PFS, progression-free survival;
DFS, disease-free survival.
*Dysplastic changes should consider the normal range of dysplastic changes (modification).
^{†}Modification to IWG response criteria.
^{‡}In some circumstances, protocol therapy may require the initiation of further treatment (e.g.,
consolidation, maintenance) before the 4-week period. Such patients can be included in the
response category into which they fit at the time the therapy is started. Transient cytopenias
during, repeated chemotherapy courses should not be considered as interrupting durability of
response, as long as they recover to the improved counts of the previous course.

The Modified International Working Group (IWG)-2006 response criteria for hematologic improvement are described in the following Table 6.

TABLE 6

| Hematologic | |
|---|---|
| improvement* | Response criteria (responses must last at least 8 wk)^{†} |
| Erythroid response | Hgb increase by ≥ 1.5 g/dL |
| (pretreatment, < 11 | Relevant reduction of units of RBC transfusions by an absolute number of |
| g/dL) | at least 4 RBC transfusions/8 wk compared with the pretreatment |
| | transfusion number in the previous 8 wk. Only RBC transfusions given |
| | for a Hgb of ≤ 9.0 g/dL pretreatment will count in the RBC transfusion |
| | response evaluation^{†} |
| Platelet response | Absolute increase of ≥ 30 × 10⁹/L for patients starting with > 20 × 10⁹/L |
| (pretreatment, < | platelets |
| 100 × 10⁹/L) | Increase from < 20 × 10⁹/L to > 20 × 10⁹/L and by at least 100%^{†} |
| Neutrophil response | At least 100% increase and an absolute increase > 0.5 × 10⁹/L^{†} |
| (pretreatment, < | |
| 1.0 × 10⁹/L) | |
| Progression or | At least 1 of the following: |
| relapse after HI^{‡} | At least 50% decrement from maximum response levels in granulocytes |
| | or platelets |
| | Reduction in Hgb by ≥ 1.5 g/dL |
| | Transfusion dependence |

Deletions to the IWG response criteria are not shown.
Hgb indicates hemoglobin;
RBC: red blood cell;
HI: hematologic improvement.
*Pretreatment counts averages of at least 2 measurements (not influenced by transfusions) ≥ 1 week apart (modification).
^{†}Modification to IWG response criteria.
^{‡}In the absence of another explanation, such as acute infection, repeated courses of
chemotherapy (modification), gastrointestinal bleeding, hemolysis, and so forth. It is
recommended that the 2 kinds of erythroid and platelet responses be reported overall as well as
by the individual response pattern.

Treatment with CPI-613 and HCQ should be continued as long as the treating physician believes there is clinical benefit, unless or until: patients exhibit disease progression; unacceptable toxicity from CPI-613 and HCQ in spite of dose reduction; patient withdrawal of consent; investigator's discretion to withdraw patients from the study because continued participation in the study is not in the patient's best interest; undercurrent illness (a condition, injury, or disease unrelated to the intended disease for which the study is investigating, that renders continuing the treatment unsafe or regular follow-up impossible); general or specific changes in the patient's condition that renders the patient ineligible for further investigational treatment; non-compliance with investigational treatment, protocol-required evaluations or follow-up visits; or termination of the clinical trial. Upon being taken off the trial, patient's survival and post-study cancer treatment will be monitored by follow up physician visits once patients are removed from trial. All patients will be followed for 5 years post treatment, or until death (unless consent for follow up withdrawn).

### CPI-613

CPI-613 is provided in 10-mL amber glass vials. Each vial contains 10 mL of CPI-613 at a concentration 50 mg/mL, equivalent to 500 mg of CPI-613. The drug product of CPI-613 is a clear and colorless solution that is free of any particulate matter.

CPI-613 is administered IV by infusion, via an IV catheter with D5W running at a rate of about 125-150 mL/hr. To avoid local reactions at and around the site of administration, CPI-613 should be administered via a central venous catheter.

CPI-613 can cause leaching of DEHP from IV infusion sets and IV bags. Therefore, DEHP-containing IV infusion sets, IV bags or syringes should not be used in mixing or administration of CPI-613. Examples of the IV sets, IV bags and syringes that do not contains DEHP and therefore can be used in the administration of CPI-613 are:
Extension Set for Syringe Pump Use: All extension sets from MED-RX do not contain DEHP.
Syringes: All Monoject syringes are DEHP free.
IV Infusion Sets: The IV infusion sets that can be used to administer CPI-613 are:
   PVC material—ADDitIV® Primary IV Set with Universal Spike, Backcheck Valve, 2 Injection Sites, DEHP-Free and Latex-Free, 15 drops/mL, REF V14453, B Braun Medical Inc.
   Latex material—Interlink® System Secondary Medication Set, 10 drops/mL, 2C7451, Baxter Healthcare Corporation
   PVC material—Surshield™ Safety Winged Infusion Set, 0.19 mL Volume, Latex-Free, DEHP-Free, SV*S25BLS, Terumo Medical Products Hangzhou Co. Ltd.
   Polyethylene material—Interlink® System Paclitaxel Set by Baxter HealthCare, Non DEHP-free: Polyethylene tubing with a 0.22 microfilter Item #2C7558 10 drops/mL
Syringes: CPI-613 drug product (50 mg/mL), and drug product diluted with D5W to various concentrations (1.6-25 mg/mL) are compatible with various types of syringes, as listed below. Therefore, any of these types of syringes, and syringes that are made with the same materials, can be used to administer CPI-613. Also, glass syringes can also be used, since glass (such as glass containers) is compatible with CPI-613 drug product.
   Norm-Ject, polyethylene barrel, polyethylene plunger, latex free (Henke Sass Wolf GMBH) syringes
   Becton Dickinson syringes
   Terumo syringes
   Monoject syringes
   Glass syringes.

CPI-613 must be diluted from 50 mg/mL to 12.5 mg/mL with 5% Dextrose Water (D5W) (i.e., 1 portion of CPI-613 diluted with 3 portions of D5W) prior to administration. The diluted drug product should be visually inspected for clarity. If haziness, precipitate or coloration (other than colorless) is observed, do not use the diluted drug product for dosing. After dilution with sterile D5W, the solution is clear and has a pH of 8.4-8.8. The diluted CPI-613 drug product has been found to be stable for 24 hrs at room temperature and refrigeration temperature.

CPI-613 must be administered IV, via an IV catheter that is free flowing and free of air in the dead space of the IV catheter, to minimize vascular irritation, inflammation and acute toxicity of CPI-613. Accidental co-administration of extra air in the dead space of IV catheters during administration of CPI-613 has demonstrated the potential to induce acute toxicity of CPI-613 according to animal studies. Also, accidental leakage of CPI-613 into the perivascular space during IV administration, which prolongs exposure of perivascular tissue to CPI-613, can induce significant local inflammation according to animal studies. To avoid local reactions at and around the site of administration, CPI-613 must be administered via a central venous catheter.

CPI-613 must not be administered as a bolus, but by infusion, at a rate of ˜0.5 mL/min, via a central venous catheter with D5W running at a rate of about 125-150 mL/hr. This is to minimize potential acute toxicity of CPI-613, according to animal studies.

The following precautions must be taken when administering CPI-613:
Confirmation of the placement of the IV line to ensure a lack of leakage of CPI-613 into the perivascular space.
Confirmation that the IV line is free flowing.
Confirmation that the IV line is free of dead air space.
Dilute CPI-613 drug product with D5W, as instructed in the study protocol.
Administer CPI-613 by infusion, not as a bolus.
After administration of CPI-613, flush the IV line with ˜10 mL of D5W to remove residual CPI-613.
To avoid local reactions at and around the site of administration, CPI-613 should be administered via a central venous catheter.

The amount of CPI-613 at each dose level is based on the BSA of the patient. The BSA values will be calculated based on the height and body weight taken during screening and this BSA value is used throughout the study. This is unless there is a >10% change in the body weight from baseline during the study. At that point, BSA should be revised based on the new body weight and height. The new BSA values will be used from that point on for the remainder of the study, unless there is another >10% change in body weight which will require another revision of the BSA.

### Other Agents

Patients cannot receive any standard or investigational treatment (except CPI-613 and hydroxychloroquine) for their MDS, or any other investigational drugs for any non-cancer indications, while on this study. All otherwise permitted concomitant medications (including trade and generic names, dosage and dosing schedule) must be recorded. Treatment of disease-related symptoms (such as nausea) is permitted. Medications administered in such instances will be considered concomitant medications and should be documented accordingly. Supportive treatment may include anti-emetic, anti-diarrhea, anti-pyretic, anti-allergic, anti-hypertensive medications, analgesics, antibiotics, allopurinol, and others such as blood products. Patients may use growth factors as per ASCO guidelines at the discretion of the treating investigator.

### Adverse Events

The CTEP Active Version of the NCI Common Terminology Criteria for Adverse Events (CTCAE 4.0) will be utilized for AE reporting. It is identified and located on the CTEP website at http://ctep.cancer.gov/protocolDevelopment/electronic_applications/ctc.htm. All appropriate treatment areas should have access to a copy of the CTEP Active Version of CTCAE.

Attribution of the Adverse Event (AE):
Definite—The AE is clearly related to the study treatment.
Probable—The AE is likely related to the study treatment.
Possible—The AE may be related to the study treatment.
Unlikely—The AE is doubtfully related to the study treatment.
Unrelated—The AE is clearly NOT related to the study treatment.

List of Adverse Events to be Reported: abdominal pain; alkaline phosphatase; ALT (SGPT); anorexia; AST (SGOT); bilirubin; (hyperbilirubinemia); calcium (hypercalcemia, hypocalcemia); creatinine; diarrhea; flushing; hemoglobin (anemia); injection site reaction; leukocytes; lymphopenia; nausea; neutrophils (neutropenia); platelets (thrombocytopenia); potassium; sodium; vomiting. All grade 3, 4, 5 adverse events should be reported on flow sheets and in ORIS regardless of whether they are on this list. All unexpected grade 4 and all grade 5 SAE's on these trials be reported for review.

Any unanticipated problems involving risks to subjects or others and adverse events shall be promptly reported to the IRB. Reporting to the IRB is required regardless of the funding source, study sponsor, or whether the event involves an investigational or marketed drug, biologic or device. Reportable events are not limited to physical injury, but include psychological, economic and social harm. Reportable events may arise as a result of drugs, biological agents, devices, procedures or other interventions, or as a result of questionnaires, surveys, observations or other interactions with research subjects.

All members of the research team are responsible for the appropriate reporting to the IRB and other applicable parties of unanticipated problems involving risk to subjects or others. The Principal Investigator, however, is ultimately responsible for ensuring the prompt reporting of unanticipated problems involving risk to subjects or others to the IRB. The Principal Investigator is also responsible for ensuring that all reported unanticipated risks to subjects and others which they receive are reviewed to determine whether the report represents a change in the risks and/or benefits to study participants, and whether any changes in the informed consent, protocol or other study-related documents are required.

Any unanticipated problems involving risks to subjects or others occurring at a site where the study has been approved by the IRB (internal events) must be reported to the IRB. Any event, incident, experience, or outcome that alters the risk versus potential benefit of the research and as a result warrants a substantive change in the research protocol or informed consent process/document in order to insure the safety, rights or welfare of research subjects must be reported to the IRB.

### Statistical Considerations

The sample size will be a total of 17 patients treated at the MTD of HCQ for this Phase ½ trial. This number is based on Simon's two stage design (Simon R., Controlled Clinical Trials, 1989, 10: 1-10) where 9 patients will be enrolled in stage 1. If none of the 9 patients have a response the study will be stopped for lack of efficacy. If one or more patients have a response the trial will continue until a total of 17 patients have been treated with the combination at the MTD of HCQ. If 2 or more of the 17 patients have a response the combination will be considered of sufficient activity to merit additional study. These parameters give an a of 0.0466 and a power of 0.8122 to detect a difference from no intervention (set to a response rate of 5%).

### Data Management and Reporting Schedule

Disease response will be assessed based on RR, PFS, and OS (as described by Cheson B. D. et al., Clinical application and proposal for modification of the International Working Group (IWG) response criteria in myelodysplasia. Blood. 108:419-425, 2006), as well as changes in the frequency of transfusion from baseline. RR and PFS, derived from hematology and bone marrow exam, will be assessed at the following specified time points:
Baseline
Week 4 of every 2 treatment cycles until cycle 6
Every 3 treatment cycles thereafter (i.e., cycle 9, cycle 12, cycle 15, etc) until evidence of disease progression.
   Bone marrow exam results should be documented at each specified time point.

### Example 15—Oral Efficacy of 6,8-Bis-Benzylthio-Octanoic Acid in Non-Small Cell Lung Cancer

Human H460 NSCLC cells were obtained from American Type Cell Culture (ATCC) (catalog no. HTB-177, Manassas, Va.). These cells tested negative for viral contamination using the Mouse Antibody Production (MAP) test, performed by Charles River Labs Molecular Division, upon the receipt of the tumor cells from ATCC. The tumor cells were maintained at 37° C. in a humidified 5% CO₂ atmosphere in T225 tissue culture flasks containing 50 mL of Roswell Park Memorial Institute (RPMI)-1640 solution with 10% Fetal Bovine Serum (FBS) and 2 mM L-glutamine. Cells were split at a ratio of 1:10 every 2-3 days by trypsinization and resuspended in fresh medium in a new flask. Cells were harvested for experiments in the same way at 70-90% confluency.

CD1-Nu/Nu female mice, ˜4-6 weeks old were obtained from Charles River Laboratories. Mice were housed 5 to a cage in a micro-isolator room in the Department of Animal Laboratory Research of New York State University (SUNY) at Stony Brook. Light-dark cycles were 12 h each daily, with light from 7 a.m. to 7 p.m. Food (Purina Rodent Chow) and water (distilled sterile-filtered water, pH 7) were provided ad libitum. Protocols and procedures were according to the rules of and approved by the SUNY Institutional Animal Care and Use Committee (IACUC).

An acclimation period of 7 days was allowed between the arrival of the animal at the study site before tumor inoculation and experimentation. Mice were inoculated subcutaneously (SC) in the right flank with 2×10⁶ human H460 NSCLC or BxPC3 pancreatic cancer cells that were suspended in 0.1 mL of Dulbeco's Phosphate Buffered Salt (PBS) solution using a 1 cc syringe with a 27-5/8 gauge needle. Tumor dimensions (length and width) were measured daily before, during and after treatment (using Vernier calipers) and the tumor volume was calculated using the prolate ellipsoid formula: (length×width²)/2. Treatment with test or control articles began 8 days post tumor cell implantation when the tumor was approximately 300 mm³.

Oral dosing of 6,8-bis-benzylthio-octanoic acid was at 100 mg/kg with 11 animals per group. 100 mg of 6,8-bis-benzylthio-octanoic acid was suspended in a small volume 0.01-0.05N NaOH in 5% dextrose and titrated to pH 7.0 with 4% Glacial Acetic Acid to 50 mg/mL. Prior to administration the suspension was diluted with 5% dextrose to 12.5 mg/mL so that the animals received 100 mg/kg with a dose volume of about 0.2 mL delivered by gastric gavage. Post tumor cell implantation, mice were treated on day 8, day 15, day 22, and day 29.

A similar study was conducted in CD-1 nude mice (n=9) inoculated with 2×10⁶ BxPC-3 cells. The study was initiated when tumors reached an average size of 150 mm³ (day 0) and CPI-613 was administered at an oral dose of 100 mg/week for 4 weeks. A comparator arm (n=9) was conducted with IP treatment at a weekly dose of 25 mg/kg.

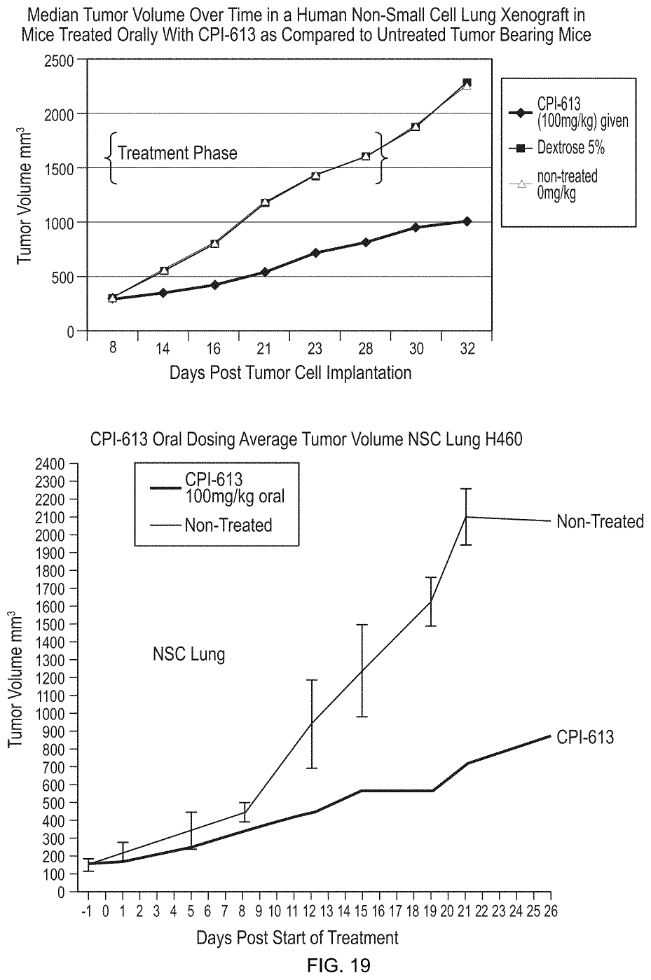

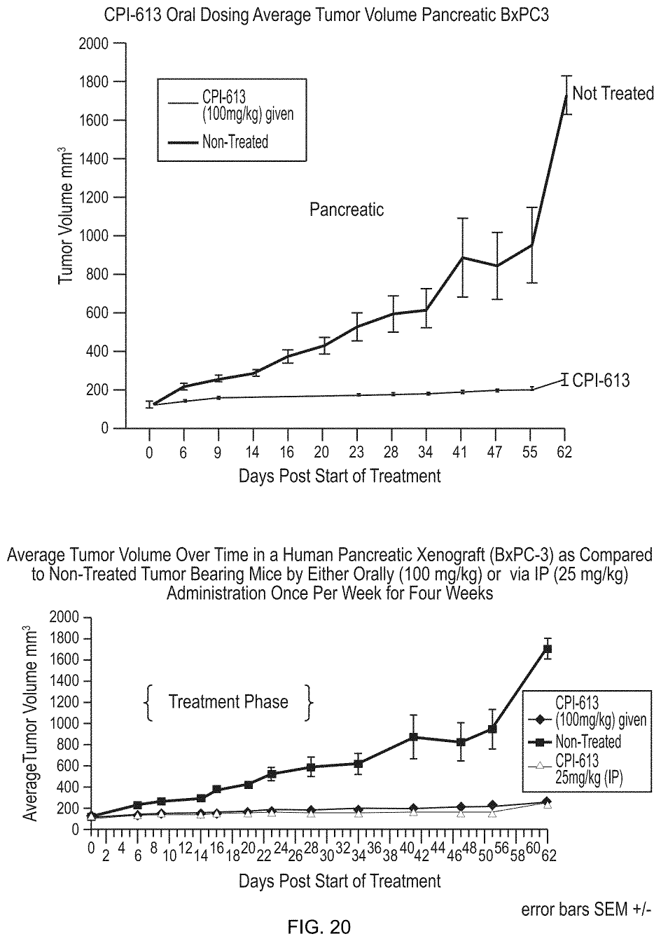

The results are presented in FIGS. 19 and 20. It is evident that the tumors in the mice treated with 6,8-bis-benzylthio-octanoic acid grew much more slowly than those in mice treated with 5% dextrose or untreated. The effect was especially pronounced in BxPC3 tumors. This example demonstrates that 6,8-bis-benzylthio-octanoic acid is effective to treat cancer when administered orally.

### Example 16—Spray Dried Dispersion Oral Formulation of 6,8-Bis-Benzylthio-Octanoic Acid

Solid amorphous dispersion formulations of 6,8-bis-benzylthio-octanoic acid (API) were prepared by mixing the API 1:4 with one of the following polymers: Eudragit L100, poly(vinylpyrrolidone) viscosity grade K30 (PVP K30), hydroxypropyl methyl cellulose (HPMC), cellulose acetate phthalate (CAP), or hydroxypropyl methylcellulose acetate succinate (HPMCAS-M), and spray drying from methanol or acetone using a small-scale Bend Lab Dryer with 35 kg/hr drying gas flow rate capacity (BLD-35). Conditions, yields, and residual solvent levels of two representative spray dried dispersion (SDD) formulations (75 g each) are presented in the following table.

| | 20% API: | 20% API: |
|---|---|---|
| Formulation | Eudragit L100 | HPMCAS-M |
| Spray Solution | 5% solids in methanol | 5% solids in acetone |
| Outlet Temp | 45° C. | 35° C. |
| Solution Feed Rate | 35 g/min | |
| Drying Gas Flow Rate | 475-500 g/min | |
| Atomization Pressure | 120 psi | |
| Nozzle | Schlick 2.0 pressure swirl atomizer | |
| Secondary Drying | 20 hr at 30° C. | |
| Dry Yield (%) | 94 | 96 |
| Residual Solvent (%) | 4.21 ± 0.02 | 1.01 ± 0.00 |
| (Wet SDD) | (MeOH) | (Acetone) |
| Residual Solvent (%) | <LOQ | <LOQ |
| (Tray-Dried Material) | | |
| API content by HPLC | 201 ± 1.1 mg/g | 198 ± 0.2 mg/g |

Scanning electron microscopy (SEM) was used to qualitatively determine particle morphology of the two SDD formulations, and to study if any degree of fusion or crystallinity was visually present. Particles show collapsed sphere morphology with no crystallization or fusion noted.

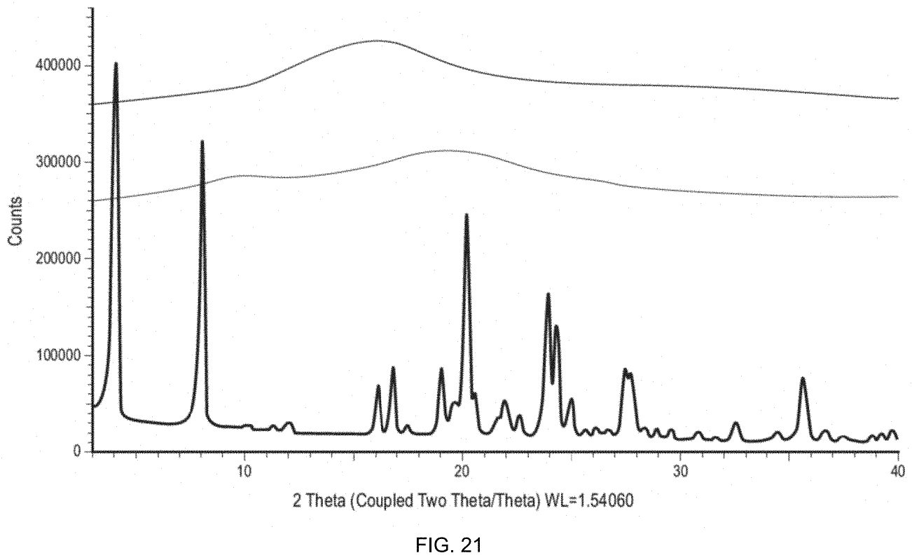

X-ray diffraction is typically sensitive to the presence of crystalline material with an LOD of about 1% of the sample mass. No crystallinity was detected by PXRD for either SDD formulation. Diffractograms in comparison to crystalline 6,8-bis-benzylthio-octanoic acid API can be found in FIG. 21, wherein the top diffractogram is the Eudragit L100 formulation, the middle diffractogram is the HPMCAS-M formulation, and the bottom diffractogram is crystalline 6,8-bis-benzylthio-octanoic acid.

### Example 17—Emulsion Oral Formulations of 6,8-Bis-Benzylthio-Octanoic Acid

Monolaurin (131 mg) and 6,8-bis-benzylthio-octanoic acid (93 mg) were warmed to 50° C. in polysorbate-80 (2.5 mL) in a round bottomed flask equipped with a magnetic stir bar. After complete dissolution to a clear solution, water (7.5 mL) was added with vigorous stirring at 50° C. to provide an emulsion.

6,8-bis-benzylthio-octanoic acid (312 mg) was combined with polysorbate 80 (6.25 g), soybean oil (1.25 g), and a lipid mix (100 mg) comprising cholesterol (14 g), cholesteryl acetate (14 g), cholesteryl benzoate (14 g), monolaurin (25.4 g), and monopalmitin (32.6 g), and the mixture heated to 50° C. until the solids dissolved (30 min). Dextrose (11.25 g) was dissolved in 236 mL of water, and the resulting aqueous dextrose solution was added to the oil solution above. The resulting two phase mixture was stirred for 30 min at rt, then vacuum filtered through a 0.22 um filter.

### Example 18—Liquid Formulations of 6,8-Bis-Benzylthio-Octanoic Acid

A 6,8-bis-benzylthio-octanoic acid solution was prepared by the steps of (a) providing a 50 mg/mL solution of 6,8-bis-benzylthio-octanoic acid in 1 M aqueous triethanolamine, and (b) diluting the 50 mg/mL solution with 5% aqueous dextrose to a concentration of 5 mg/mL. The resulting 5 mg/mL solution is identified as “18A” in Example 19 below.

A suspension vehicle was prepared by the steps of: (a) combining tris buffer (48 mg) and HPMCAS-HF (20 mg) in 14 mL of distilled water, (b) adjusting the pH to 7.4 with dilute sodium hydroxide to dissolve the HPMCAS-HF, (c) heating the resulting solution to approximately 90° C., (d) adding Methocel A4M Premium (100 mg) to the hot solution, (e) stirring the mixture vigorously to suspend the undissolved Methocel A4M, (f) cooling and stirring the mixture with an ice bath until the Methocel A4M dissolves (approximately 10 minutes), (g) diluting the solution with distilled/deionized water to bring the total volume to 20 mL, and (h) adjusting the pH to 7.4 with dilute acetic acid or dilute sodium hydroxide to provide the suspension vehicle.

Suspensions of the spray-dried formulations of Example 16 were prepared by adding 400 mg of the respective SDD formulation to a mortar, slowly adding 4 mL of the suspension vehicle (mixing thoroughly with a pestle after each small addition to uniformly disperse), and then transferring to a flask and stirring for one minute prior to administration. The resulting suspension of the Eudragit L100 SDD formulation (20 mg/mL 6,8-bis-benzylthio-octanoic acid) is identified as “18B” in Example 19 below. The resulting suspension of the HPMCAS-M SDD formulation (20 mg/mL 6,8-bis-benzylthio-octanoic acid) is identified as “18C” in Example 19 below.

In the same way, a 20 mg/mL suspension of 6,8-bis-benzylthio-octanoic acid was prepared by adding 80 mg 6,8-bis-benzylthio-octanoic acid to a mortar, slowly adding 4 mL of the suspension vehicle (mixing thoroughly with a pestle after each small addition to uniformly disperse), and then transferring to a flask and stirring for one minute prior to administration. The resulting suspension of 6,8-bis-benzylthio-octanoic acid is identified as “18D” in Example 19 below. A solution of 6,8-bis-benzylthio-octanoic acid was prepared by dissolving SOLUTOL® (polyoxyl 15 hydroxystearate; KOLLIPHOR® HS 15) (3 grams) in distilled water (7 mL) to form a 30% solution, adding 6,8-bis-benzylthio-octanoic acid (50 mg) to 5 mL of the 30% solution, vortexing for 1 minute, and then sonicating for 45 minutes to provide a clear colorless solution (10 mg/mL; pH 7). The resulting solution is identified as “18E” in Example 19 below.

### Example 19—Oral Bioavailability of 6,8-Bis-Benzylthio-Octanoic Acid

Six groups of 16 BALB/c nude mice (8 males and 8 females) per group were administered 6,8-bis-benzylthio-octanoic acid in six different ways: (1) 5 μL/g IV injection (tail vein) of the triethanolamine/dextrose aqueous solution of Example 18 (25 mg/kg; 5 mL/kg; Ex. 18A); (2) 5 μL/g IP injection of the triethanolamine/dextrose aqueous solution of Example 18 (25 mg/kg; 5 mL/kg; 18A); (3) 5 μL/g oral administration of the Eudragit L100 SDD suspension of Example 18 (100 mg/kg; 5 mL/kg; 18B); (4) 5 μL/g oral administration of the HPMCAS-M SDD suspension of Example 18 (100 mg/kg; 5 mL/kg; 18C); (5) 5 μL/g oral administration of the 20 mg/mL 6,8-bis-benzylthio-octanoic acid suspension of Example 18 (100 mg/kg; 5 mL/kg; 18D); or (6) 10 μL/g oral administration of the 10 mg/mL SOLUTOL solution of Example 18 (100 mg/kg; 10 mL/kg; 18E). In each experiment, about 80 μL of blood was collected from one subgroup of 4 male and 4 female mice at 0.083, 1, 4, and 24 hours after dosing, and from the other subgroup of 4 male and 4 female mice at 0.5, 2, and 8 hours. Plasma from the collected blood samples was analyzed by LC-MS/MS for the presence of 6,8-bis-benzylthio-octanoic acid.

| | | | | Bioavail- | AUC | | | T |
|---|---|---|---|---|---|---|---|---|
| | | | Dose | ability | Last | | | ½ |
| Formulation | Route | Mice (n) | (mg/kg) | (%) | (uM * hr) | Cmax | Tmax | (hr) |
| 18A | IV | 16 | 25 | — | 36 | 92 | 0.08 | 2.0 |
| (TEA/dextrose) | | | | | | | | |
| 18A | IP | 16 | 25 | 83 | 29 | 103 | 0.08 | 3.9 |
| (TEA/dextrose) | | | | | | | | |
| 18B (Eudragit | PO | 16 | 100 | 44 | 61 | 94 | 0.08 | 2.0 |
| SDD) | | | | | | | | |
| 18C (HPMCAS-M | PO | 16 | 100 | 43 | 60 | 69 | 0.08 | 1.1 |
| SDD) | | | | | | | | |
| 18D (CPI-613) | PO | 16 | 100 | 57 | 82 | 82 | 0.50 | 3.7 |
| 18E (Solutol) | PO | 16 | 100 | 127 | 175 | 229 | 0.08 | 4.4 |

This example demonstrates that 6,8-bis-benzylthio-octanoic acid is orally bioavailable.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating cancer in a human patient in need thereof, comprising the step of administering to the patient a therapeutically effective amount of 6,8-bis-benzylthio-octanoic acid and an autophagy inhibitor.

2. The method of claim 1, wherein the cancer is characterized by displaying increased autophagy in response to contact with CPI-613.

3. The method of claim 1, wherein the cancer is a lymphoma.

4. The method of claim 3, wherein the lymphoma is relapsed or refractory Hodgkin lymphoma.

5. The method of claim 4, wherein the patient has failed brentuximab vedotin and a PD-1 inhibitor.

6. The method of claim 3, wherein the lymphoma is relapsed or refractory T-cell non-Hodgkin lymphoma.

7. The method of claim 6, further comprising the step of administering to the patient a therapeutically effective amount of bendamustine hydrochloride.

8. The method of claim 3, wherein the lymphoma is relapsed or refractory Burkitt's lymphoma.

9. The method of claim 3, wherein the lymphoma is high-grade B-cell lymphoma with rearrangements of MYC and BCL2 and/or BCL6.

10. The method of claim 1, wherein the cancer is a leukemia.

11. The method of claim 1, wherein the cancer is a carcinoma.

12. The method of claim 1, wherein the cancer is a sarcoma.

13. The method of claim 1, wherein the cancer is a myeloma.

14. The method of claim 1, wherein the cancer is a clear cell cancer.

15. The method of claim 14, wherein the cancer is a clear cell sarcoma.

16. The method of claim 14, wherein the cancer is a clear cell carcinoma.

17. The method of claim 16, wherein the cancer is a clear cell renal carcinoma.

18. The method of claim 1, wherein the cancer is a solid tumor.

19. The method of claim 1, wherein the cancer is a brain or spinal cord cancer.

20. The method of claim 1, wherein the cancer is a melanoma.

21. The method of claim 1, wherein the cancer is a blastoma.

22. The method of claim 1, wherein the cancer is a germ cell tumor.

23. The method of claim 1, wherein the cancer is a cancer of the pancreas.

24. The method of claim 1, wherein the cancer is a cancer of the prostate.

25. The method of claim 1, wherein the cancer is a myelodysplastic syndrome.

26. The method of claim 25, wherein the cancer is high risk myelodysplastic syndrome.

27. The method of claim 26, wherein the cancer is high risk myelodysplastic syndrome in a patient who has failed to respond, progressed, or relapsed while on hypomethylating therapy

28. The method of any preceding claim, wherein the autophagy inhibitor is a 4-aminoquinoline.

29. The method of claim 28, wherein the autophagy inhibitor is hydroxychloroquine.

30. The method of claim 29, wherein the hydroxychloroquine is administered to the patient as hydroxychloroquine sulfate.

31. The method of claim 28, wherein the autophagy inhibitor is chloroquine.

32. The method of claim 31, wherein the chloroquine is administered to the patient as chloroquine phosphate.
